(19) United States
(12) Patent Application Publication
(10) Pub. No.: US 2024/0382528 A1
(43) Pub. Date: Nov. 21, 2024

(54) TREATMENT OF POLYCYTHEMIA VERA VIA CRISPR/AAV6 GENOME EDITING

(71) Applicant: The Board of Trustees of the Leland Stanford Junior University, Stanford, CA (US)

(72) Inventors: Matthew H. Porteus, Stanford, CA (US); Michael Kyle Cromer, Stanford, CA (US)

(73) Assignee: The Board of Trustees of the Leland Stanford Junior University, Stanford, CA (US)

(21) Appl. No.: 18/696,014

(22) PCT Filed: Oct. 4, 2022
(86) PCT No.: PCT/US2022/077505

§ 371 Date: Mar. 27, 2024

Related U.S. Application Data
(60) Provisional application No. 63/252,540, filed on Oct. 5, 2021.

(51) Int. Cl. A61K 35/28; A61P 35/00; C12N 5/0789; C12N 9/22; C12N 15/11; C12N 15/86; C12N 15/90

(52) U.S. Cl. CPC A61K 35/28; A61P 35/00; C12N 5/0647; C12N 9/22; C12N 15/111; C12N 15/86; C12N 15/907; C12N 2310/20; C12N 2310/321; C12N 2510/00; C12N 2750/14143

(57) ABSTRACT

The present disclosure provides methods and compositions for genetically modifying hematopoietic stem and progenitor cells (HSPCs), in particular by correcting a JAK2^{V617F} mutation in the HSPCs.

### CROSS-REFERENCE TO RELATED APPLICATIONS

This application claims priority to U.S. Provisional Application No. 63/252,540, filed Oct. 5, 2021, the disclosure of which is hereby incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant No. HL135607 awarded by the National Institutes of Health. The Government has certain rights in the invention.

### BACKGROUND

Polycythemia very (PV) is a type of blood cancer that affects approximately 50 out of every 100,000 individuals. PV results from the overproduction of red blood cells (RBCs) by the bone marrow, causing the blood to thicken and slowing its flow. PV causes a dramatic increase in the likelihood of adverse circulatory events such as blood clots, strokes, and heart attacks. PV exists in a congenital form that severely shortens the life of affected individuals, as well as a sporadic form that often appears in individuals over the age of 60.

In the large majority of cases, PV is caused by a mutation in the JAK2 gene called JAK2^{V617F}. The JAK2 (Janus kinase 2) gene encodes a non-receptor tyrosine kinase (also called JAK2, as used herein, JAK2 or JAK2^{V617} written in italics refers to a nucleic acid such as a gene, allele, locus, construct, or polynucleotide sequence, and JAK2 or JAK2^{V617} not written in italics refers to a protein) that is involved in cytokine and growth factor signaling. JAK2 kinase associates with the erythropoietin (EPO) receptor and STAT transcription factors, and in the absence of ligand binding JAK2 is inactive and not phosphorylated. Upon binding of EPO to the receptor, however. JAK2 becomes phosphorylated and activated, which in turn leads to the activation of STAT proteins and resulting gene expression. The JAK2^{V617F} mutation leads to constitutive JAK2 activation wherein the kinase is active even in the absence of EPO binding, resulting in accelerated proliferation and differentiation of erythroid cells.

The current options for PV treatment include compounds such as small molecule JAK2 inhibitors (e.g., ruxolitinib), myeloablation (e.g., using busulfan), cell proliferation inhibitors such as hydroxvurea, or the direct removal of RBCs by bloodletting. Such options are less than ideal, however, having numerous drawbacks related to safety, efficacy, convenience, and potential side effects.

There is thus a need for new, safe and effective approaches for reducing or preventing the production of excess RBCs in subjects with conditions such as polycythemia vera. The present disclosure satisfies this need and provides other advantages as well.

### BRIEF SUMMARY

In one aspect, the present disclosure provides a method of genetically modifying a hematopoietic stem and progenitor cell (HSPC) comprising a JAK2^{V617F} mutation from a subject, the method comprising: introducing into the HSPC an RNA-guided nuclease, a donor template, and a mutation-specific guide RNA that specifically hybridizes to a mutant JAK2 polynucleotide comprising a JAK2^{V617F} mutation, but does not hybridize to a wild-type JAK2 polynucleotide lacking the JAK2^{V617F} mutation; wherein the donor template comprises a corrective JAK2 nucleotide sequence that comprises a wild-type sequence at the position of the JAK2^{V617F} mutation, flanked by a first homology arm corresponding to a JAK2 genomic sequence located upstream of the JAK2^{V617F} mutation and a second homology arm corresponding to a JAK2 genomic sequence located downstream of the JAK2^{V617F} mutation; wherein the RNA-guided nuclease cleaves a mutant JAK2^{V617F} gene in the genome of the cell but does not cleave a wild-type JAK2 gene in the genome of the cell; and wherein the cleaved mutant JAK2^{V617F} gene is modified by integrating the corrective JAK2 nucleotide sequence into the genome by homology directed repair (HDR), thereby eliminating the JAK2^{V617F} mutation from the genome and generating a genetically modified HSPC.

In some embodiments, the method further comprises isolating the HSPC from the subject prior to introducing the RNA-guided nuclease, the donor template, and the first guide RNA into the cell. In some embodiments, the method further comprises introducing into the HSPC a second guide RNA comprising a sequence that specifically hybridizes to a target site within an intron in the JAK2 gene. In some embodiments, the intron in the JAK2 gene is located between exons 12 and 13. In some embodiments, the second homology arm corresponds to a JAK2 genomic sequence located downstream of the target site of the second guide RNA. In some embodiments, the first homology arm comprises the nucleotide sequence of SEQ ID NO:1 or a subsequence thereof, or a sequence comprising at least about 70%, 75%, 80%, 85%, 90%, 95%, 96%, 97%, 98%, 99%, or more identity to SEQ ID NO:1 or a subsequence thereof. In some embodiments, the second homology arm comprises the nucleotide sequence of SEQ ID NO:2 or a subsequence thereof, or a sequence comprising at least about 70%, 75%, 80%, 85%, 90%, 95%, 96%, 97%, 98%, 99%, or more identity to SEQ ID NO:2 or a subsequence thereof. In some embodiments, the donor template comprises a portion of exon 12 downstream of the site corresponding to the JAK2^{V617F} mutation and all of exons 13-23 of the wild-type JAK2 gene. In some embodiments, the donor template comprises SEQ ID NO:4, or a sequence comprising at least about 70%, 75%, 80%, 85%, 90%, 95%, 96%, 97%, 98%, 99%, or more identity to SEQ ID NO:4. In some embodiments, the corrective JAK2 nucleotide sequence comprises a JAK2 3′ UTR.

In some embodiments, the JAK2 3′ UTR comprises the sequence of SEQ ID NO:5 or a subsequence thereof, or a sequence comprising at least about 70%, 75%, 80% 85%, 90%, 95%, 96%, 97%, 98%, 99%, or more identity to SEQ ID NO:5 or a subsequence thereof. In some embodiments, the mutation-specific guide RNA specifically hybridizes to a polynucleotide comprising the nucleotide sequence of SEQ ID NO:9, and does not specifically hybridize to a polynucleotide comprising the nucleotide sequence of SEQ ID NO:8. In some embodiments, the mutation-specific guide RNA specifically guides an RNA-guided nuclease to cleave a polynucleotide comprising the nucleotide sequence of SEQ ID NO:9, and does not specifically guide an RNA-guided nuclease to cleave a polynucleotide comprising the nucleotide sequence of SEQ ID NO:8. In some embodiments, the target sequence of the second guide RNA comprises the nucleotide sequence of any one of SEQ ID NOS:10-15. In some embodiments, the target sequence of the second guide RNA comprises the nucleotide sequence of SEQ ID NO: 15. In some embodiments, the guide RNA comprises one or more 2′-O-methyl-3′-phosphorothioate (MS) modifications. In some embodiments, the one or more 2′-O-methyl-3′-phosphorothioate (MS) modifications are present at the three terminal nucleotides of the 5′ and 3′ ends of the guide RNA. In some embodiments, the RNA-guided nuclease is Cas9. In some embodiments, the Cas9 is a High Fidelity Cas9. In some embodiments, the mutation-specific and/or second guide RNA and the RNA-guided nuclease are introduced into the HSPC as a ribonucleoprotein (RNP) complex by electroporation. In some embodiments, the donor template is introduced into the HSPC using a recombinant adeno-associated virus (rAAV) vector. In some embodiments, the rAAV vector is a AAV6 vector.

In some embodiments, the method reduces the proliferation and/or erythropoietic differentiation of the genetically modified HSPC as compared to an HSPC into which the guide RNA, the RNA-guided nuclease, and/or the donor template has not been introduced. In some embodiments, the subject has polycythemia vera (PV). In some embodiments, the genetically modified HSPC is reintroduced into the subject. In some embodiments, the reintroduction of the genetically modified HSPC ameliorates one or more symptoms of PV. In some embodiments, the subject is a human.

In another aspect, the present disclosure provides a method for treating polycythemia vera in a subject in need thereof, the method comprising administering any of the herein-described genetically modified HSPCs to the subject, wherein the genetically modified HSPC engrafts in the subject and replaces all or a portion of the JAK2^{V617F} mutant HSPCs or other cells in the erythroid lineage.

In another aspect, the present disclosure provides a genetically modified HSPC comprising a corrective JAK2 nucleotide sequence, wherein the genetically modified HSPC is generated using any of the herein-described methods.

In another aspect, the present disclosure provides a donor template comprising a homology region comprising SEQ ID NO: 1 or SEQ ID NO:2 or a subsequence thereof, or a nucleotide sequence comprising at least about 70%, 75%, 80%, 85%, 90%, 95%, 96%, 97%, 98%, 99% or more identity to SEQ ID NO:1 or SEQ ID NO:2 or a subsequence thereof.

In another aspect, the present disclosure provides a donor template a nucleotide sequence comprising SEQ ID NO:7 or a subsequence thereof, or a sequence comprising at least about 70%, 75%, 80%, 85%, 90%, 95%, 96%, 97%, 98%, 99%, or more identity to SEQ ID NO:7 or a subsequence thereof.

In another aspect, the present disclosure provides a transgene comprising a corrective JAK2 nucleotide sequence, wherein the nucleotide sequence comprises the sequence of SEQ ID NO:4 or a nucleotide sequence comprising at least about 70%, 75%, 80%, 85%, 90%, 95%, 96%, 97%, 98%, 99% or more identity to SEQ ID NO:4.

In another aspect, the present disclosure provides a guide RNA that specifically hybridizes to a polynucleotide comprising the nucleotide sequence of SEQ ID NO:9, but does not specifically hybridize to a polynucleotide comprising the nucleotide sequence of SEQ ID NO:8.

In another aspect, the present disclosure provides a guide RNA comprising a target sequence comprising any one of SEQ ID NOS:10-15 (e.g., SEQ ID NO:15), or a sequence comprising 1, 2, or 3 mismatches with any one of SEQ ID NOS: 10-15 (e.g., SEQ ID NO: 15).

In another aspect, the present disclosure provides a ribonucleoprotein (RNP) complex comprising: (a) an RNA-guided nuclease; and (b) a guide RNA that specifically hybridizes to a polynucleotide comprising the nucleotide sequence of SEQ ID NO:9, but does not specifically hybridize to a polynucleotide comprising the nucleotide sequence of SEQ ID NO:8, and/or a guide RNA comprising a target sequence comprising any one of SEQ ID NOS:10-15 (e.g., SEQ ID NO:15).

In another aspect, the present disclosure provides an HSPC comprising any of the herein described donor templates, transgenes, guide RNAs, and/or RNP complexes.

### BRIEF DESCRIPTION OF THE DRAWINGS

FIG. 1: One possible guide site near JAK2^{V617F} mutation. For the SpCas9 ortholog, a nearby NGG PAM sequence is required for a guide sequence to overlap the JAK2^{V617F} mutation (G>T missense mutation).

FIGS. 2A-2C: We designed WT- and mutant-overlapping guides and found that our WT guide created ˜50% indels whereas the mutant guide sequence (distinguished by only a single altered nucleotide 5 bp from the PAM) induced no detectable cutting (FIG. 2A). To induce a higher frequency of mutant-specific deletion, we designed gRNAs in the following intron that may be paired as a two-guide system (FIGS. 2B-2C). We found that by combining Cas9 RNP pre-complexed with WT guide and guides 4 and 6, we were able to induce a high degree of deletions between the predicted cleavage sites of each gRNA (FIG. 2B).

FIG. 3: Using a 2-guide system (WT gRNA paired with sg4 or sg6), we screened homology arms to identify those that best mediated integration of a custom cassette. Version 1 (top panel) is a more conventional homology arm scheme for homology-directed repair following a Cas9-induced DNA break—with homology anus immediately flanking the disease-causing mutation. Version 2 (bottom panel) is a homology arm scheme where the left HA corresponds to the DNA sequence immediately upstream of the leftmost guide's cut site while the right HA corresponds to the DNA sequence immediately downstream of the rightmost guide's cut site.

FIGS. 4A-4D: To test the alternative homology arm schemes for their ability to mediate targeted integration, we designed constitutive SFFV-GFP-polyA vectors (FIGS. 4A-4B) to rapidly identify editing frequencies by flow cytometry (FIG. 4C). We found that all 2-guide editing schemes were more effective than the single WT gRNA paired with the cut-site (“CS”) integration scheme (FIG. 4D).

FIGS. 5A-5C: ddPCR was used to quantify editing frequencies of our SFFV-GFP-polyA vectors in three separate healthy HSPC donors. We also used primers outside of the homology arms to amplify the edited locus and excised the band that did not undergo knock-in (FIG. 5A). We then Sanger sequenced these bands and quantified indels using ICE (ice.synthcgo.com). Percentages of homologous recombination (HR)+indel alleles are shown above each column in FIG. 5B and summarized in FIG. 5C.

FIG. 6: Clinical correction vectors that either: 1) directly correct the V617F mutation; 2) introduce the remaining JAK2 cDNA following the mutation; or 3) the remaining JAK2 cDNA followed by a constitutive PGK-tNGFR selection cassette. These vectors use a mutant-overlapping guide along with sg6 and have homology arms that immediately flank the V617F mutation. div.=divergence (i.e., introducing silent mutations at every possible codon to disguise homology to the endogenous gene so that the entire cassette integrates).

FIG. 7: To determine the specificity of Cas9 for the mutant vs. wild-type (WT) allele, we generated a homozygous JAK2^{V617F} iPSC line. We then tested the WT and mutant gRNAs (SEQ ID NOS:17 and 18, respectively) on both WT and mutant iPSC lines. Both guides displayed extreme specificity for the appropriate allele with no detectable cleavage in the WT line using the mutant gRNA.

FIG. 8: We next tested whether the dual-gRNA strategy is able to boost JAK2 deactivation above the single mutant-specific gRNA approach. When paired with sg6, editing efficiency for both WT gRNA in WT iPSCs and mutant (MT) gRNA in mutant iPSCs was dramatically improved.

FIG. 9: Schematic of an exemplary repair donor and resulting edited allele in accordance with an embodiment of the present disclosure. The repair donor both corrects the JAK2^{V617F} mutation and then includes all downstream exons (12-23) of the wild-type JAK2 gene as a codon-diverged and codon-optimized cDNA (i.e., to disguise homology to the wild-type gene and thus prevent premature homologous recombination).

### DETAILED DESCRIPTION

### 1. Introduction

The present disclosure provides methods and compositions for correcting mutations in genomic sequences, in particular mutations in the JAK2 gene, in, e.g., hematopoietic stem and progenitor cells (HSPCs).

The present methods can be used to specifically correct the JAK2^{V617F} mutation in a cell without impairing a wild-type JAK2 gene. In particular, the present disclosure provides guide RNA sequences that specifically recognize the mutant form of the JAK2 gene, while not substantially binding to (and/or guiding the RNA-guided nuclease to cleave) the wild-type form, enabling the cleavage of the JAK2^{V617F} gene by an RNA-directed nuclease such as Cas9 but leaving the wild-type copy intact. By cleaving JAK2^{V617F} in the presence of a donor template comprising a polynucleotide encoding wild-type JAK2, the wild-type coding sequence can integrate into the genome at the site of cleavage by homology directed recombination (HDR), thereby correcting the JAK2^{V617F} mutation.

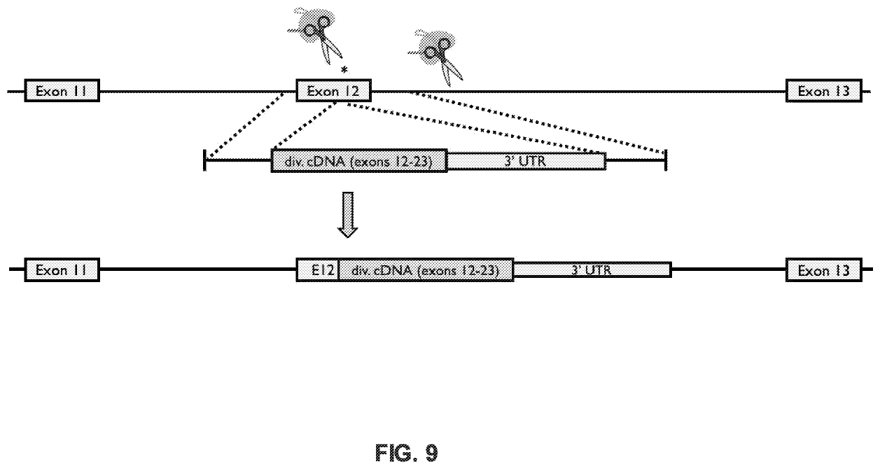

In some embodiments, a second guide RNA is also used that specifically targets a sequence within an intron downstream of the JAK2^{V617F} mutation. See, e.g., FIG. 9. The inclusion of a second guide RNA can increase the frequency of deletions in the JAK2^{V617F} mutant locus, which can increase the rate of integration of the wild-type JAK2 coding sequence in turn. While the second guide RNA can also target a wild-type copy of the gene and potentially induce small insertions or deletions (indels), these are not expected to affect the expression of the wild-type JAK2 protein due to their location in an intron.

When only one guide RNA is used (i.e., a guide RNA targeting the JAK2^{V617F} mutation, but with no guide RNA targeting a downstream intron), the homology arms in the donor template can correspond to (i.e., be homologous to) sequences on either side of the guide RNA target site. When a second, intron-targeting guide RNA is also used, in some embodiments the homology arms in the donor template are split, i.e., non-contiguous, so that the left arm starts at or around the mutation site (i.e., target sequence of the first guide RNA) and runs upstream, and the right arm starts at or around the target site within the intron (i.e., target sequence of the second guide RNA) and runs downstream.

In particular embodiments, the present methods are used to correct a JAK2^{V617F} mutation in HSPCs from a subject with polycythemia vera or another myeloproliferative disorder caused by the JAK2^{V617F} mutation. For example, HSPCs obtained from a subject with one mutant JAK2 allele and one wild-type allele can be genetically modified using the present methods to correct the mutant allele such that the modified (corrected) cell thereafter has two wild-type alleles. The modified cells can then be reintroduced into the patient, e.g., in conjunction with the removal of mutant erythroid lineage cells from the subject, to allow the corrected cells to proliferate normally in the subject and produce healthy numbers of RBCs.

### 2. General

Practicing this disclosure utilizes routine techniques in the field of molecular biology. Basic texts disclosing the general methods of use in this disclosure include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### 3. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs. and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081(1991) Ohtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

As used herein, a first polynucleotide or polypeptide is “heterologous” to an organism or a second polynucleotide or polypeptide sequence if the first polynucleotide or polypeptide originates from a foreign species compared to the organism or second polynucleotide or polypeptide, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “expression” and “expressed” refer to the production of a transcriptional and/or translational product, e.g., of a JAK2 cDNA, transgene, or encoded protein. In some embodiments, the term refers to the production of a transcriptional and/or translational product encoded by a gene or a portion thereof. The level of expression of a DNA molecule in a cell may be assessed on the basis of either the amount of corresponding mRNA that is present within the cell or the amount of protein encoded by that DNA produced by the cell.

“Conservatively modified variants” applies to both amino acid and nucleic acid sequences. With respect to particular nucleic acid sequences. “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a colon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants of a protein can have an increased stability, assembly, or activity as described herein.

The following eight groups each contain amino acids that are conservative substitutions for one another:
1) Alanine (A), Glvcine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins, W. H. Freeman and Co., N.Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more pol nucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93%, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J. Mol. Biol. 215: 403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues, always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W. T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sd. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR-Cas” system refers to a class of bacterial systems for defense against foreign nucleic acids. CRISPR-Cas systems are found in a wide range of bacterial and archaeal organisms. CRISPR-Cas systems fall into two classes with six types, I, II, III, IV, V, and VI as well as many sub-types, with Class 1 including types I and III CRISPR systems, and Class 2 including types II, IV, V and VI, Class 1 subtypes include subtypes I-A to I-F, for example. See, e.g., Fonfara et al., Nature 532, 7600 (2016); Zetschc et al., Cell 163, 759-771 (2015); Adli et al. (2018). Endogenous CRISPR-Cas systems include a CRISPR locus containing repeat clusters separated by non-repeating spacer sequences that correspond to sequences from viruses and other mobile genetic elements, and Cas proteins that carry out multiple functions including spacer acquisition, RNA processing from the CRISPR locus, target identification, and cleavage. In class 1 systems these activities are effected by multiple Cas proteins, with Cas3 providing the endonuclease activity, whereas in class 2 systems they are all carried out by a single Cas, Cas9.

A “homologous repair template” or “donor template” refers to a polynucleotide sequence that can be used to repair a double stranded break (DSB) in the DNA, e.g., a CRISPR/Cas9-mediated break at a JAK2 locus as induced using the herein-described methods and compositions. The homologous repair template comprises homology to the genomic sequence surrounding the DSB, i.e., comprising JAK2 homology arms. In particular embodiments, two distinct homologous regions are present on the template, with each region comprising at least 50,100,200, 300, 400, 500, 600, 700, 800, 900, 400-1000, 500-900, or more nucleotides of homology with the corresponding genomic sequence. The repair template can be present in any form, e.g., on a plasmid that is introduced into the cell, as a free floating doubled stranded DNA template (e.g., a template that is liberated from a plasmid in the cell), or as single stranded DNA. In particular embodiments, the template is present within a viral vector, e.g., an adeno-associated viral vector such as AAV6.

As used herein, “homologous recombination” or “FIR” refers to insertion of a nucleotide sequence during repair of double-strand breaks in DNA via homology-directed repair mechanisms. This process uses a “donor template” or “homologous repair template” with homology to nucleotide sequence in the region of the break as a template for repairing a double-strand break. The presence of a double-stranded break facilitates integration of the donor sequence. The donor sequence may be physically integrated or used as a template for repair of the break via homologous recombination, resulting in the introduction of all or part of the nucleotide sequence. This process is used by a number of different gene editing platforms that create the double-strand break, such as meganucleases, such as zinc finger nucleases (ZFNs), transcription activator-like effector nucleases (TALENs), and the CRISPR-Cas9 gene editing systems. In particular embodiments, HR involves double-stranded breaks induced by CRISPR-Cas9.

JAK2 (Janus kinase 2) encodes a non-receptor tyrosine kinase involved in cytokine and growth factor signaling. The JAK2 kinase associates with the erythropoietin receptor and STAT transcription factors, and ervthropoietin (EPO) binding to the receptor leads to JAK2 phosphorylation and activation, which in turn leads to the activation of STAT proteins and to gene expression. The JAK2^{V617F} mutation leads to constitutive activation and EPO independence of the kinase, resulting in accelerated proliferation and differentiation of cells of the erythroid lineage, and leading in turn to myeloproliferative disorders such as polycythemia vera. The NCBI gene ID for human JAK2 is 3717, and the UniProt ID for human JAK2 is 060674, the entire disclosures of which are herein incorporated by reference. An exemplary partial JAK2 coding sequence (i.e., a cDNA sequence from the middle of exon 12, near the location of the JAK2^{V617F} mutation through exon 23) is shown herein as SEQ ID NO:4. As used herein, JAK2 can refer to any nucleotide sequence comprising about 70%, 75%, 80%, 85%, 90%, 95%, 96%, 97%, 98%, 99%, or more homology to SEQ ID NO:4 or a subsequence therein, or to any nucleotide sequence encoding the JAK2 protein (e.g., as disclosed in Uniprot ID 060674) or a fragment thereof, or any protein comprising at least about 70%, 75%, 80%, 85%, 90%, 95%, 96%, 97%, 98%, 99%, or more homology to the JAK2 protein or a fragment thereof. A JAK2 gene or JAK2 protein can refer to a wild-type gene or protein or to a mutant gene or protein such as JAK2^{V617F}/JAK2^{V617F}. As used herein. JAK2^{V617F} refers to the protein comprising the valine to phenylalanine substitution, and JAK2^{V617F} refers to the gene or polynucleotide encoding the mutant protein.

“Polycythemia vera” or “PV” is a type of blood cancer caused by the overproduction of red blood cells by the bone marrow. The excess red blood cells thickens the blood and increases the likelihood of blood clots, strokes, and heart attacks. PV (and other myeloproliferative disorders) is caused by a mutation in the JAK2 gene (the JAK2^{V617F} mutation, in which the valine at position 617 in the JAK2 protein is replaced by phenylalanine). The mutant JAK2^{V617F} form of the protein is constitutively active and EPO independent, leading to the rapid proliferation and differentiation of cells in the erythroid lineage. Traditional treatments for PV include treatment with small molecule inhibitors such as ruxolitinib, myeloablation (e.g., using busulfan), treatment with hydroxvurea, or bloodletting. The present methods allow the correction of the JAK2^{V617F} mutation in cells, restoring the wild-type function and EPO dependence of the cells.

As used herein, the terms “hematopoietic stem and progenitor cell” and “HSPC” refer to a hematopoietic stem cell (HSC), a hematopoietic progenitor cell (HPC), or a population of hematopoietic stem cells and hematopoietic progenitor cells.

### 4. CRISPR/Cas Systems Specifically Targeting the JAK2^{V617F} Mutation

The present disclosure is based in part on the identification of CRISPR guide sequences that specifically direct the cleavage of the mutant JAK2^{V617F} allele (i.e., the allele encoding the JAK2^{V617F} protein) by RNA-guided nucleases, while sparing the wild-type allele from guide RNA-guided cleavage. In particular embodiments, a second guide RNA is used that targets an intron downstream of the JAK2^{V617F} mutation. Accordingly, the present disclosure provides a CRISPR/AAV6-mediated genome editing method that can achieve high rates of targeted integration of wild-type JAK2-coding sequences specifically at mutant JAK2^{V617F} loci. The integrated wild-type coding sequence eliminates the presence of the mutant allele from the cell, resulting in the restoration of wild-type JAK2 function and EPO dependence in the kinase. Cells edited at this locus are capable of long-term engraftment and hematopoietic reconstitution.

sgRNAs

The single guide RNAs (sgRNAs) of the present disclosure target the JAK2 locus. Certain sgRNAs used in the present methods specifically target the JAK2^{V617F} mutant allele, while not targeting wild-type JAK2 alleles. Other sgRNAs used in the present methods target intron sequences downstream of the JAK2^{V617F} mutation in both wild-type and mutant loci. sgRNAs interact with a site-directed nuclease such as Cas9 and specifically bind to or hybridize to a target nucleic acid within the genome of a cell, such that the sgRNA and the site-directed nuclease co-localize to the target nucleic acid in the genome of the cell. The sgRNAs as used herein comprise a targeting sequence comprising homology (or complementarity) to a target DNA sequence at the JAK2 locus, and a constant region that mediates binding to Cas9 or another RNA-guided nuclease.

In particular embodiments, the sgRNA targets a sequence comprising the sequences shown as SEQ ID NO:9, or a sequence having, e.g., at least 90%, 91%, 92%, 93%, 94%, 95%, 96%, 97%, 98%, 99%, or more identity to, e.g., comprising 1, 2, 3, or more nucleotide substitutions, additions, or subtractions relative to, SEQ ID NO:9, so long that the target sequence comprises a thymine base at a position corresponding to nucleotide 16 in SEQ ID NO:9. In particular embodiments, the sgRNA specifically hybridizes to a polynucleotide comprising the nucleotide sequence of SEQ ID NO:9, and does not specifically hybridize to a polynucleotide comprising the nucleotide sequence of SEQ ID NO:8. In particular embodiments, the sgRNA specifically guides an RNA-guided nuclease to cleave a polynucleotide comprising the nucleotide sequence of SEQ ID NO:9, and does not specifically guide an RNA-guided nuclease to cleave a polynucleotide comprising the nucleotide sequence of SEQ ID NO:8.

In some embodiments, a second (or more) sgRNA is used that targets an intron downstream of the JAK2^{V617F} mutation. In particular embodiments, the targeted intron sequence is located between exon 12 and exon 13. In some embodiments, the target sequence of such intron-targeting sgRNAs comprises a target sequence of any one of SEQ ID NOS 10-15, or a sequence having, e.g., at least 90%, 91%, 92%, 93%, 94%, 95%, 96%, 97%, 98%, 99%, or more identity to, e.g., comprising 1, 2, 3, or more nucleotide substitutions, additions or subtractions relative to any of, SEQ ID NOS: 10-15. In particular embodiments, the target sequence of the intron-targeting sgRNA comprises SEQ ID NO: 13 or 15, or a sequence having, e.g., at least 90%, 91%, 92%, 93%, 94%, 95%, 96%, 97%, 98%, 99%, or more identity to, e.g., comprising 1, 2, 3, or more nucleotide substitutions, additions or subtractions relative to SEQ ID NO:13 or 15.

The targeting sequence of the sgRNAs may be, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or 15-25, 18-22, or 19-21 nucleotides in length, and shares homology with a targeted genomic sequence, in particular at a position adjacent to a CRISPR PAM sequence. The sgRNA targeting sequence is designed to be homologous to the target DNA, i.e., to share the same sequence with the non-bound strand of the DNA template or to be complementary to the strand of the template DNA that is bound by the sgRNA. The homology or complementarity of the targeting sequence can be perfect (i.e., sharing 100% homology or 100% complementarity to the target DNA sequence) or the targeting sequence can be substantially homologous (i.e., having less than 100% homology or complementarity, e.g., with 1-4 mismatches with the target DNA sequence).

Each sgRNA also includes a constant region that interacts with or binds to the site-directed nuclease, e.g., Cas9. In the nucleic acid constructs provided herein, the constant region of an sgRNA can be from about 70 to 250 nucleotides in length, or about 75-100 nucleotides in length, 75-85 nucleotides in length, or about 80-90 nucleotides in length, or 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length. The overall length of the sgRNA can be, e.g., from about 80-300 nucleotides in length, or about 80-150 nucleotides in length, or about 80-120 nucleotides in length, or about 90-110 nucleotides in length, or, e.g, 90, 91, 92, 93, 94, 95, 96, 97, 98, 99, 100, 101, 102, 103, 104, 105, 106, 107, 108, 109, or 110 nucleotides in length.

It will be appreciated that it is also possible to use two-piece gRNAs (cr:tracrRNAs) in the present methods, i.e., with separate crRNA and tracrRNA molecules in which the target sequence is defined by the crispr RNA (crRNA), and the tracrRNA provides a binding scaffold for the Cas nuclease.

In some embodiments, the sgRNAs comprise one or more modified nucleotides. For example, the polynucleotide sequences of the sgRNAs may also comprise RNA analogs, derivatives, or combinations thereof. For example, the probes can be modified at the base moiety, at the sugar moiety, or at the phosphate backbone (e.g., phosphorothioates). In some embodiments, the sgRNAs comprise 3 phosphorothiate internucleotide linkages, 2′-O-methyl-3′-phosphoacetate modifications, 2′-fluoro-pyrimidines, S-constrained ethyl sugar modifications, or others, at one or more nucleotides. In particular embodiments, the sgRNAs comprise 2′-O-methyl-3′-phosphorothioate (MS) modifications at one or more nucleotides (see, e.g., Hendel et al. (2015) Nat. Biotech. 33(9):985-989, the entire disclosure of which is herein incorporated by reference). In particular embodiments, the 2′-O-methyl-3′-phosphorothioate (MS) modifications are at the three terminal nucleotides of the 5′ and 3′ ends of the sgRNA.

The sgRNAs can be obtained in any of a number of ways. For sgRNAs, primers can be synthesized in the laboratory using an oligo synthesizer, e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher, Biolytic. IDT, Sigma-Aldritch, GeneScript, etc.

### RNA-Guided Nucleases

Any CRISPR-Cas nuclease can be used in the method, i.e., a CRISPR-Cas nuclease capable of interacting with a guide RNA and cleaving the DNA at the target site as defined by the guide RNA. In some embodiments, the nuclease is Cas9 or Cpf1. In particular embodiments, the nuclease is Cas9. The Cas9 or other nuclease used in the present methods can be from any source, so long that it is capable of binding to an sgRNA as described herein and being guided to and cleaving the specific JAK2^{V617F} sequence or JAK2 intron sequence targeted by the sgRNA. In particular embodiments, the Cas9 is from Streptococcus pyogenes. In particular embodiments, the Cas9 is a high fidelity Cas9 (e.g., a high-fidelity SpCas9 variant as described in Vakulskas, et al., Nature Medicine (2018)).

Also disclosed herein are CRISPR/Cas or CRISPR/Cpf1 systems that target and cleave DNA at the JAK2 locus. An exemplary CRISPR/Cas system comprises (a) a Cas (e.g., Cas9) or Cpf1 polypeptide or a nucleic acid encoding said polypeptide, and (b) an sgRNA that hybridizes specifically to JAK2^{V617F}, and optionally a second sgRNA that hybridizes specifically to a JAK2 intron), or a nucleic acid encoding said guide RNA. In some instances, the nuclease systems described herein, further comprises a donor template as described herein. In particular embodiments, the CRISPR/Cas system comprises an RNP comprising an sgRNA targeting JAK2^{V617F} and a Cas protein such as Cas9.

In addition to the CRISPR/Cas9 platform (which is a type II CRISPR/Cas system), alternative systems exist including type I CRISPR/Cas systems, type III CRISPR/Cas systems, and type V CRISPR/Cas systems. Various CRISPR/Cas9 systems have been disclosed, including Streptococcus pyogenes Cas9 (SpCas9), Streptococcus thermophilus Cas9 (StCas9). Campylobacter jejuni Cas9 (CjCas9) and Neisseria cinerea Cas9 (NcCas9) to name a few. Alternatives to the Cas system include the Francisella novicida Cpf1 (FnCpf1), Acidaminococcus sp. Cpf1 (AsCpf1), and Lachnospiraceae bacterium ND2006 Cpf1 (LbCpf1) systems. Any of the above CRISPR systems may be used to induce a single or double stranded break at the JAK2^{V617F} mutation and optionally JAK2 intron to carry out the methods disclosed herein.

Introducing the sgRNA and Cas Protein into Cells

The guide RNA and nuclease can be introduced into the cell using any suitable method, e.g., by introducing one or more polynucleotides encoding the guide RNA and the nuclease into the cell, e.g., using a vector such as a viral vector or delivered as naked DNA or RNA, such that the guide RNA and nuclease are expressed in the cell. In some embodiments, one or more polynucleotides encoding the sgRNA, the nuclease or a combination thereof are included in an expression cassette. In some embodiments, the sgRNA, the nuclease, or both sgRNA and nuclease are expressed in the cell from an expression cassette. In some embodiments, the sgRNA, the nuclease, or both sgRNA and nuclease are expressed in the cell under the control of a heterologous promoter. In some embodiments, one or more polynucleotides encoding the sgRNA and the nuclease are operatively linked to a heterologous promoter. In particular embodiments, the guide RNA and nuclease are assembled into ribonucleoproteins (RNPs) prior to delivery to the cells, and the RNPs are introduced into the cell by, e.g., electroporation. RNPs are complexes of RNA and RNA-binding proteins. In the context of the present methods, the RNPs comprise the RNA-binding nuclease (e.g., Cas9) assembled with the guide RNA (e.g., sgRNA), such that the RNPs are capable of binding to the target DNA (through the gRNA component of the RNP) and cleaving it (via the protein nuclease component of the RNP). As used herein, an RNP for use in the present methods can comprise any of the herein-described guide RNAs and any of the herein-described RNA-guided nucleases.

Animal cells, mammalian cells, preferably human cells, modified ex vivo, in vitro, or in vivo are contemplated. Also included are cells of other primates; mammals, including commercially relevant mammals, such as cattle, pigs, horses, sheep, cats, dogs, mice, rats; birds, including commercially relevant birds such as poultry, chickens, ducks, geese, and/or turkeys.

In some embodiments, the cell is an embryonic stem cell, a stem cell, a progenitor cell, a pluripotent stem cell, an induced pluripotent stem (iPS) cell, a somatic stem cell, a differentiated cell, a mesenchymal stem cell or a mesenchymal stromal cell, a neural stem cell, a hematopoietic stem cell or a hematopoietic progenitor cell, an adipose stem cell, a keratinocyte, a skeletal stem cell, a muscle stem cell, a fibroblast, an NIA cell, a B-cell, a T cell, or a peripheral blood mononuclear cell (PBMC). In particular embodiments, the cells are CD34⁺ hematopoietic stem and progenitor cells (HSPCs), e.g., cord blood-derived (CB), adult peripheral blood-derived (PB), or bone marrow derived HSPCs.

HSPCs can be isolated from a subject, e.g., by collecting mobilized peripheral blood and then enriching the HSPCs using the CD34 marker. In some embodiments, the cells are from a subject with polycythemia vera or another myeloproliferative disorder. In some embodiments, a method is provided of treating a subject with polycythemia vera, comprising genetically modifying a plurality of HSPCs isolated from the subject so as to correct a JAK2^{V617F} mutation in the cells, and reintroducing the HSPCs into the subject. In some such embodiments, HSPCs differentiate into red blood cells (RBCs) in vivo, and when mutant RBCs and other cells of the erythroid lineage are removed, the corrected RBCs are present at normal, non-cancerous levels.

To avoid immune rejection of the modified cells when administered to a subject, the cells to be modified are preferably derived from the subject's own cells. Thus, preferably the mammalian cells are autologous cells from the subject to be treated with the modified cells. In some embodiments, however, the cells are allogeneic, i.e., isolated from an HLA-matched or HLA-compatible, or otherwise suitable, donor.

In some embodiments, cells are harvested from the subject and modified according to the methods disclosed herein, which can include selecting certain cell types, optionally expanding the cells and optionally culturing the cells, and which can additionally include selecting cells that contain the corrected JAK2^{V617F} mutation. In particular embodiments, such modified cells are then reintroduced into the subject.

Further disclosed herein are methods of using said nuclease systems to produce the modified host cells described herein, comprising introducing into the cell (a) an RNP of the present disclosure that targets and cleaves DNA at a JAK2^{V617F} mutation, and optionally at a JAK2 intron downstream of the JAK2^{V617} mutation, and (b) a homologous donor template or vector as described herein. Each component can be introduced into the cell directly or can be expressed in the cell by introducing a nucleic acid encoding the components of said one or more nuclease systems.

Such methods will target integration of the functional JAK2 coding sequence at the endogenous JAK2^{V617F} locus in a host cell ex vivo. Such methods can further comprise (a) introducing a donor template or vector into the cell, optionally after expanding said cells, or optionally before expanding said cells, and (b) optionally culturing the cell.

In some embodiments, the disclosure herein contemplates a method of producing a modified mammalian host cell, the method comprising introducing into a mammalian cell: (a) an RNP comprising a Cas nuclease such as Cas9 and an sgRNA specific to the JAK2^{V617F} allele, and (b) a homologous donor template or vector as described herein.

In any of these methods, the nuclease can produce one or more single stranded breaks within the JAK2^{V617F} gene, or a double-stranded break within the JAK2^{V617F} gene. In these methods, the JAK2^{V617F} gene is modified by homologous recombination with said donor template or vector to result in insertion of the wild-type JAK2 coding sequence into the locus. The methods can further comprise (c) selecting cells that contain the wild-type coding sequence integrated into the JAK2^{V617F} locus.

In some embodiments, i53 (Canny et al. (2018) Nat Biotechnol 36:95) is introduced into the cell in order to promote integration of the donor template by homology directed repair (HDR) versus integration by non-homologous end joining (NHEJ). For example, an mRNA encoding i53 can be introduced into the cell, e.g., by electroporation at the same time as an sgRNA-Cas9 RNP. The sequence of i53 can be found, inter aha, at www.addgene.org/92170/sequences/.

Techniques for the insertion of transgenes, including large transgenes, capable of expressing functional proteins, including enzymes, cytokines, antibodies, and cell surface receptors are known in the art (See, e.g. Bak and Porteus, Cell Rep. 2017 Jul. 18; 20(3): 750-756 (integration of EGFR); Kanojia et al., Stem Cells. 2015 Oct.; 33(10)2985-94 (expression of anti-Her2 antibody): Eyquem et al., Nature. 2017 Mar. 2; 543(7643):113-117 (site-specific integration of a CAR); O'Connell et al., 2010 PLoS ONE 5(8): e12009 (expression of human IL-7); Tuszynski et al., Nat Med. 2005 May; 11(5):551-5 (expression of NGF in fibroblasts); Sessa et al., Lancet. 2016 Jul. 30:388(10043):476-87 (expression of arylsulfatase A in ex vivo gene therapy to treat MLD), Rocca et al., Science Translational Medicine 25 Oct. 2017: Vol. 9, Issue 413, eaaj2347 (expression of frataxin); Bak and Porteus, Cell Reports, Vol. 20, Issue 3, 18 Jul. 2017. Pages 750-756 (integrating large transgene cassettes into a single locus), Dever et al., Nature 17 Nov. 2016: 539, 384-389 (adding tNGFR into hematopoietic stem cells (HSC) and HSPCs to select and enrich for modified cells); each of which is herein incorporated by reference in its entirety.

### Homologous Repair Templates

The wild-type JAK2 coding sequence to be integrated, which is comprised by a polynucleotide or donor construct, can be any polynucleotide that does not contain a mutation encoding the JAK2^{V617F} isoform and whose gene product can provide functional JAK2 activity and normal EPO dependence in red blood cells and other cells of the erythroid lineage. For example, the coding sequence can comprise JAK2 sequences around the site of the JAK2^{V617F} mutation, e.g., comprising exon 12. In some embodiments, the coding sequence comprises the JAK2 sequence around the mutation in exon 12, as well as some or all of the exons downstream of exon 12, e.g., from exon 12 (or a portion of exon 12) up to and including exon 13, exon 14, exon 15, exon 16, exon 17, exon 18, exon 19, exon 20, exon 21, exon 22, or exon 23. In particular embodiments, the coding sequence comprises the sequence shown as SEQ ID NO:4 or a subsequence thereof, or a sequence comprising at least about 70%, 75%, 80%, 85%, 90%, 95%, 96%, 97%, 98%, 99%, or more identity to SEQ ID NO:4 or a subsequence thereof.

In some embodiments, the integrated sequence comprises one or more silent mutations surrounding the Cas9 cut site, which can help eliminate cutting following homologous recombination and integration of the wild-type coding sequence. In some such embodiments, the integrated sequence comprises the sequence shown as SEQ ID NO:7 or a subsequence thereof, or a sequence comprising at least about 70%, 75%, 80%, 85%, 90%, 95%, 96%, 97%, 98%, 99%, or more identity to SEQ ID NO:7 or a subsequence thereof.

The integrated coding sequence (i.e., transgene) comprises all or a portion of a functional coding sequence for JAK2, with optional elements such as introns, WPREs, polyA regions, UTRs (e.g. 5′ or 3′ UTRs). The optional elements can be from any source. In some embodiments, the integrated sequence comprises a JAK2 3′ UTR, e.g., the sequence shown as SEQ ID NO:5 or a subsequence thereof, or a sequence comprising at least about 70%, 75%, 80%, 85%, 90%, 95%, 96%, 97%, 98%, 99%, or more identity to SEQ ID NO:5 or a subsequence thereof.

In some embodiments, the JAK2 coding sequence in the homologous repair template is codon-optimized, e.g., comprises at least about 70%, 75%, 80%, 85%, 90%, 95%, or more homology to the corresponding wild-type coding sequence or cDNA, or a fragment thereof.

In some embodiments, the template comprises a polyA sequence or signal, e.g., a bovine growth hormone polyA sequence or a rabbit beta-globin polyA sequence, at the 3′ end of the cDNA. In some embodiments, a Woodchuck Hepatitis Virus Posttranscriptional Regulatory Element (WPRE) is included within the 3′UTR of the template, e.g., between the 3 end of the the coding sequence and the 5′ end of the polyA sequence, so as to increase the expression of the transgene. Any suitable WPRE sequence can be used; See, e.g., Zufferey et al. (1999) J. Virol. 73(4):2886-2892; Donello, et al. (1998). J Virol 72: 5085-5092; Loeb, et al. (1999). Hum Gene Ther 10: 2295-2305; the entire disclosures of which are herein incorporated by reference).

To facilitate homologous recombination, the transgene is flanked within the polynucleotide or donor construct by sequences homologous to the target genomic sequence. In particular embodiments, the transgene is flanked by sequences adjacent to the one or more cleavage sites of cleavage as defined by the guide RNA or RNAs. For example, in some embodiments in which a single guide RNA is used to cleave the JAK2^{V617F} allele (i.e., without a second, intron-targeting guide RNA), the transgene is flanked by one sequence (referred to as a “left homology arm”) that is homologous to the region 5′ to the guide RNA target sequence (i.e., starting at or around the target sequence and running upstream) and a second sequence (referred to as a “right homology arm”) that is homologous to the region 3′ of the guide RNA target sequence (i.e., starting at or around the target sequence and running downstream). Accordingly, upon cleavage and repair of the DNA break using the homologous repair template, the region of the cleaved JAK2 locus surrounding the guide RNA target sequence (which comprises the JAK2^{V617F} mutation) is replaced by the wild-type JAK2 coding sequence, starting with a single CRISPR-Cas9-mediated cleavage event. In particular embodiments, the JAK2 left homology arm comprises the sequence shown as SEQ ID NO:1 or a subsequence thereof, or to a sequence comprising at least about 70%, 75%, 80°%, 85%, 90%, 95%, 96%, 97%, 98%, 99% or more homology to SEQ ID NO:1 or a subsequence thereof. In particular embodiments, the JAK2 right homology arm comprises the sequence shown as SEQ ID NO:2 or a subsequence thereof, or to a sequence comprising at least about 70%, 75%, 80%, 85%, 90%, 95%, 96%, 97%, 98%, 99% or more homology to SEQ ID NO:2 or a subsequence thereof. In particular embodiments, the wild-type coding sequence replaces the mutant JAK2^{V617F} coding sequence (i.e., replaces at least the portion of the JAK2 gene comprising the JAK2^{V617F} mutation) such that the expression of the wild-type JAK2 gene is driven by the endogenous JAK2 promoter.

In embodiments in which a second guide RNA is used, i.e., a first (i.e., mutation-specific) guide RNA targeting the JAK2^{V617F} mutation and a second guide RNA targeting a downstream intron (such as the intron between exons 12 and 13), typically a left homology arm is used that is homologous to the region 5′ to the first guide RNA target sequence (i.e., starting at or around the target sequence, or mutation site, and running upstream), and a right homology arm is used that either is homologous to the region 3′ of the first guide RNA target sequence (i.e., starting at or around the target sequence, or mutation site, and running downstream) or is homologous to the region 3′ of the second (intronic) guide RNA sequence (i.e., starting at or around the intronic guide RNA target sequence and running downstream). For example, in particular embodiments: (i) a left homology arm is used that comprises the sequence shown as SEQ ID NO:1 or a subsequence thereof, or to a sequence comprising at least about 70%, 75%, 80%, 85%, 90%, 95%, 96%, 97%, 98%, 99% or more homology to SEQ ID NO:1 or a subsequence thereof; and (ii) a right homology arm is used that comprises either (a) the sequence shown as SEQ ID NO:2 or a subsequence thereof, or a sequence comprising at least about 70%, 75%, 80%, 85%, 90%, 95%, 96%, 97%, 98%, 99% or more homology to SEQ ID NO:2 or a subsequence thereof, or (b) the sequence shown as SEQ ID NO:3 or a subsequence thereof, or to a sequence comprising at least about 70%, 75%, 80%, 85%, 90%, 95%, 96%, 97%, 98%, 99% or more homology to SEQ ID NO:3 or a subsequence thereof.

In some embodiments, a part or a fragment of the mutant JAK2 gene (i.e., a portion comprising at least the JAK2^{V617F} mutation) is replaced by the integrated wild-type JAK2 coding sequence (i.e., transgene). In some embodiments, the whole coding sequence of the mutant JAK2 gene is replaced by the integrated wild-type coding sequence (i.e., transgene). In some embodiments, all or part of the mutant JAK2 coding sequence (or transgene) and one or more regulatory sequences of the JAK2 gene is replaced by the integrated sequence. In some embodiments, the target mutant JAK2 gene sequence replaced by the integrated transgene comprises an open reading frame. In some embodiments, the target mutant JAK2 gene sequence replaced by the transgene comprises an expression cassette. In some embodiments, the target mutant JAK2 gene sequence replaced by the transgene comprises a sequence that transcribes into a wild-type JAK2 precursor mRNA. In some embodiments, the target mutant JAK2 gene sequence replaced by the transgene comprises a 5′UTR, one or more introns, one or more exons, and a 3′ UTR.

In some embodiments, the 5′ (or left) homology arm is at least 100 bp, 200 bp, 300 bp, 400 bp, 500 bp, 600 bp, 700 bp, 800 bp, 900 bp, 1000 bp or more in length. In some embodiments, the, the 5′ homology arm is 100 bp, 150 bp, 200 bp, 250 bp, 275 bp, 300 bp. 325 bp, 350 bp, 375 bp, 400 bp, 450 bp, or greater than 500 bp in length. In some embodiments, the 5′ homology arm is at least 400 bp in length. In some embodiments, the 5′ homology arm is at least 500 bp, 600 bp, 700 bp, 800 bp, 900 bp, or 1000 bp in length. In some embodiments, the 5′ homology arm is at least 850 bp in length. In some embodiments, the 5′ homology arm is 400-500 bp. In some embodiments, the 5′ homology arm is 400-500 bp, 400-550 bp, 400-600 bp, 400-650 bp, 400-700 bp, 400-750 bp, 400-800 bp, 400-850 bp, 400-900 bp, 400-950 bp, 400-1000 bp, 400-1100 bp, 400-1200 bp, 400-1300 bp, 400-1400 bp, 450-500 bp, 450-550 bp, 450-600 bp, 450-650 bp, 450-700 bp, 450-750 bp, 450-800 bp, 450-850 bp, 450-900 bp, 450-950 bp, 450-1000 bp, 450-1100 bp, 450-1200 bp, 450-1300 bp, 450-1450 bp, 500-600 bp, 500-650 bp, 500-700 bp, 500-750 bp, 500-800 bp, 500-850 bp, 500-900 bp, 500-950 bp, 500-1000 bp, 500-1100 bp, 500-1200 bp, 500-1300 bp, 500-1500 bp, 550-600 bp, 550-650 bp, 550-700 bp, 550-750 bp, 550-800 bp, 550-850 bp, 550-900 bp, 550-950 bp, 550-1000 bp, 550-1100 bp, 550-1200 bp, 550-1300 bp, 550-1500 bp, 600-650 bp, 600-700 bp, 600-750 bp, 600-800 bp, 600-850 bp, 600-900 bp, 600-950 bp, 600-1000 bp, 600-1100 bp, 600-1200 bp, 600-1300 bp, 600-1600 bp, 650-700 bp, 650-750 bp, 650-800 bp, 650-850 bp, 650-900 bp, 650-950 bp, 650-1000 bp, 650-1100 bp, 650-1200 bp, 650-1300 bp, 650-1500 bp, 700-700 bp, 700-750 bp, 700-800 bp, 700-850 bp, 700-900 bp, 700-950 bp, 700-1000 bp, 700-1100 bp, 700-1200 bp, 700-1300 bp, 700-1500 bp, 750-800 bp, 750-850 bp, 750-900 bp, 750-950 bp, 750-1000 bp, 750-1100 bp, 750-1200 bp, 750-1300 bp, 750-1500 bp, 800-850 bp, 800-900 bp, 800-950 bp, 800-1000 bp, 800-1100 bp, 800-1200 bp, 800-1300 bp, 800-1500 bp, 850-900 bp, 850-950 bp, 850-1000 bp, 850-1100 bp, 850-1200 bp, 850-1300 bp, 850-1500 bp, 900-950 bp, 900-1000 bp, 900-1100 bp, 900-1200 bp, 900-1300 bp, 900-1500 bp, 1000-1100 bp, 1100-1200 bp, 1200-1300 bp, 1300-1400 bp, or 1400-1500 bp in length. In particular embodiments, the 5′ homology arm is about 900 nucleotides in length.

In some embodiments, the 3′ (or right) homology arm is at least 100 bp, 200 bp, 300 bp, 400 bp, 500 bp, 600 bp, 700 bp, 800 bp, 900 bp, 1000 bp or more in length. In some embodiments, the, the 3′ homology arm is 100 bp, 150 bp, 200 bp, 250 bp, 275 bp, 300 bp, 325 bp, 350 bp, 375 bp, 400 bp, 450 bp, or greater than 500 bp in length. In some embodiments, the 3′ homology arm is at least 400 bp in length. In some embodiments, the 3′ homology arm is at least 500 bp, 600 bp, 700 bp, 800 bp, 900 bp, or 1000 bp in length. In some embodiments, the 3′ homology arm is at least 850 bp in length. In some embodiments, the 3′ homology arm is 400-500 bp. In some embodiments, the 3′ homology arm is 400-500 bp. 400-550 bp, 400-600 bp, 400-650 bp, 400-700 bp, 400-750 bp, 400-800 bp, 400-850 bp, 400-900 bp, 400-950 bp, 400-1000 bp, 400-1100 bp, 400-1200 bp, 400-1300 bp, 400-1400 bp, 450-500 bp, 450-550 bp, 450-600 bp, 450-650 bp, 450-700 bp, 450-750 bp, 450-800 bp, 450-850 bp, 450-900 bp, 450-950 bp, 450-1000 bp, 450-1100 bp, 450-1200 bp, 450-1300 bp, 450-1450 bp, 500-600 bp, 500-650 bp, 500-700 bp, 500-750 bp, 500-800 bp, 500-850 bp, 500-900 bp, 500-950 bp, 500-1000 bp, 500-1100 bp, 500-1200 bp, 500-1300 bp, 500-1500 bp, 550-600 bp, 550-650 bp, 550-700 bp, 550-750 bp, 550-800 bp, 550-850 bp, 550-900 bp, 550-950 bp, 550-1000 bp, 550-1100 bp, 550-1200 bp, 550-1300 bp, 550-1500 bp, 600-650 bp, 600-700 bp, 600-750 bp, 600-800 bp, 600-850 bp, 600-900 bp, 600-950 bp, 600-1000 bp, 600-1100 bp, 600-1200 bp, 600-1300 bp, 600-1600 bp, 650-700 bp, 650-750 bp, 650-800 bp, 650-850 bp, 650-900 bp, 650-950 bp, 650-1000 bp, 650-1100 bp, 650-1200 bp, 650-1300 bp, 650-1500 bp, 700-700 bp, 700-750 bp, 700-800 bp, 700-850 bp, 700-900 bp, 700-950 bp, 700-1000 bp, 700-1100 bp, 700-1200 bp, 700-1300 bp, 700-1500 bp, 750-800 bp, 750-850 bp, 750-900 bp, 750-950 bp, 750-1000 bp, 750-1100 bp, 750-1200 bp, 750-1300 bp, 750-1500 bp, 800-850 bp, 800-900 bp, 800-950 bp, 800-1000 bp, 800-1100 bp, 800-1200 bp, 800-1300 bp, 800-1500 bp, 850-900 bp, 850-950 bp, 850-1000 bp, 850-1100 bp, 850-1200 bp, 850-1300 bp, 850-1500 bp, 900-950 bp, 900-1000 bp, 900-1100 bp, 900-1200 bp, 900-1300 bp, 900-1500 bp, 1000-1100 bp, 1100-1200 bp, 1200-1300 bp, 1300-1400 bp, or 1400-1500 bp in length. In particular embodiments, the 3′ homology arm is about 900 or about 1057 nucleotides in length.

Any suitable method can be used to introduce the polynucleotide, or donor construct, into the cell. In particular embodiments, the polynucleotide is introduced using a recombinant adeno-associated viral vector (rAAV). For example, the rAAV can be from serotype 1 (e.g., an rAAV1 vector), 2 (e.g., an rAAV2 vector), 3 (e.g., an rAAV3 vector), 4 (e.g., an rAAV4 vector), 5 (e.g., an rAAV5 vector), 6 (e.g., an rAAV6 vector), 7 (e.g., an rAAV7 vector), 8 (e.g., an rAAV8 vector), 9 (e.g., an rAAV9 vector), 10 (e.g., an rAAV10 vector), or 11 (e.g., an rAAV11 vector). In particular embodiments, the vector is an rAAV6 vector. In some instances, the donor template is single stranded, double stranded, a plasmid or a DNA fragment. In some instances, plasmids comprise elements necessary for replication, including a promoter and optionally a 3′ UTR.

Further disclosed herein are vectors comprising (a) one or more nucleotide sequences homologous to the JAK2 locus, and (b) a wild-type JAK2 coding sequence as described herein. The vector can be a viral vector, such as a retroviral, lentiviral (both integration competent and integration defective lentiviral vectors), adenoviral, adeno-associated viral or herpes simplex viral vector. Viral vectors may further comprise genes necessary for replication of the viral vector.

In some embodiments, the targeting construct comprises: (1) a viral vector backbone, e.g. an AAV backbone, to generate virus; (2) arms of homology to the target site of at least 200 bp but ideally at least 400 bp or at least 900 on each side to assure high levels of reproducible targeting to the site (see. Porteus, Annual Review of Pharmacology and Toxicology, Vol. 56:163-190 (2016); which is hereby incorporated by reference in its entirety); (3) a coding sequence encoding at least the portion of a functional JAK2 protein encompassing the JAK2^{V617F} mutation; and optionally (4) an additional marker gene to allow for enrichment and/or monitoring of the modified host cells. Any AAV known in the art can be used. In some embodiments the primary AAV serotype is AAV6. In some embodiments, the vector, e.g., rAAV6 vector, comprising the donor template is from about 1-2 kb, 2-3 kb, 3-4 kb, 4-5 kb. 5-6 kb, 6-7 kb, 7-8 kb, or larger. In some embodiments, the targeting construct comprises at least one sequence selected from the group consisting of SEQ ID NOS:1, 2, 3, 4, 5, and 7, or at least one sequence comprising at least about 70°i°, 75%, 80% a 85%, 90%, 95%, 963%, 97%, 98%, 99% or more homology to any one of SEQ ID NOS: 1, 2, 3, 4, 5, or 7.

Suitable marker genes are known in the art and include Myc, HA, FLAG, GFP, truncated NGFR, truncated EGFR, truncated CD20, truncated CD19, as well as antibiotic resistance genes. In some embodiments, the homologous repair template and/or vector (e.g., AAV6) comprises an expression cassette comprising a coding sequence for truncated nerve growth factor receptor (tNGFR), operably linked to a promoter such as the Ubiquitin C promoter.

The inserted construct can also include other safety switches, such as a standard suicide gene into the locus (e.g. iCasp9) in circumstances where rapid removal of cells might be required due to acute toxicity. The present disclosure provides a robust safety switch so that any engineered cell transplanted into a body can be eliminated, e.g., by removal of an auxotrophic factor. This is especially important if the engineered cell has transformed into a cancerous cell.

The present methods allow for the efficient integration of the donor template at the endogenous mutant JAK2^{V617F} allele. In some embodiments, the present methods allow for the insertion of the donor template in 20%, 25%, 30%, 35%, 40%, 45%, 50%, 55%, 60%, 65%, or more cells, e.g., cells from an individual with polycythemia vera. The methods also allow for the production of genetically modified, EPO-dependent HSPC or erythroid lineage cells, e.g., cells from an individual with polycythemia vera. In some embodiments, the genetically modified cells produced using the present methods display increased EPO dependence relative to a JAK2^{V617F} mutant cell that has not been genetically modified, i.e., has not been contacted with an sgRNA. RNA-dependent nuclease, and/or donor template as described herein. In some embodiments, the genetically modified cells produced using the present methods display a substantially similar level of EPO dependence relative to a wild-type HSPC or other erythroid cell, i.e. a cell without the JAK2^{V617F} mutation. In some embodiments, the genetically modified cells produced using the present methods display decreased rates of proliferation and/or erythroid differentiation (in the presence or absence of EPO) relative to a JAK2^{V617F} mutant cell that has not been genetically modified, i.e., has not been contacted with an sgRNA, RNA-dependent nuclease, and/or donor template as described herein. In some embodiments, the genetically modified cells produced using the present methods display a substantially similar rate of proliferation and/or erythroid differentiation relative to a wild-type HSPC or other erythroid cell, i.e. a cell without the JAK2^{V617F} mutation. In some embodiments, the genetically modified cells are outcompeted in a common culture in vitro (i.e., do not proliferate as rapidly as, and occupy progressively lower percentage of the total cells in the culture as compared to) JAK2^{V617F} mutant cells that have not been genetically modified, i.e., have not been contacted with an sgRNA. RNA-dependent nuclease, and/or donor template as described herein. In some embodiments, the proliferation of the the genetically modified cells is, e.g., 5%, 10%, 15%, 20%, 25%, 30%, 35%, 40%, 45%, 50%, or more slower than that of JAK2^{V617F} mutant cells that have not been genetically modified, i.e., have not been contacted with an sgRNA, RNA-dependent nuclease, and/or donor template as described herein.

In some embodiments, the CRISPR-mediated systems as described herein (e.g., comprising a guide RNA, RNA-guided nuclease, and homologous repair template) are assessed in primary HSPCs, e.g., as derived from mobilized peripheral blood or from cord blood. In such embodiments, the HSPCs can be WT primary HSPCs (e.g., for initial testing of the system) or from patient-derived HSPCs (e.g., for pre-clinical in vitro testing).

### 5. Methods of Treatment

Following the integration of the wild-type JAK2 sequence into the genome of the HSPC and confirming the correction of the encoded therapeutic protein (e.g., confirming that no EPO-independent mutant JAK2 protein is present in the cell), a plurality of modified HSPCs can be reintroduced into the subject. In some embodiments, the HSPCs are introduced by intrafemoral injection, such that they can populate the bone marrow and differentiate into, e.g., red blood cells. In some embodiments, the HSPCs are introduced by intravenous injection. In particular embodiments, mutant (i.e. non-corrected) cells of the erythroid lineage would be removed from the patient prior to the introduction of the corrected HSPCs to clear out the HSC niche in the bone marrow, such that the genetically modified HSPCs can replace all or a substantial part of the mutant cells in the subject. In particular, the body of residual mutant HSCs are removed, and then the hematopoietic system is repopulated with approximately 100% corrected HSCs. Removal of the non-corrected erythroid lineage cells can be removed by, e.g., myeloablation with drugs such as busulfan. In some embodiments, antibody-based conditioning regimens are used prior to transplantation of corrected HSCs.

Disclosed herein, in some embodiments, are methods of treating a genetic disorder, e.g., polycythemia vera in an individual in need thereof, the method comprising providing to the individual an autologous (or allogeneic) genetically corrected cell using the genome modification methods disclosed herein. In some instances, the method comprises a modified host cell ex vivo, comprising a corrected JAK2^{V617F} mutation, wherein the modified host cell no longer expresses the JAK2^{V617F} mutant form of the protein.

### Pharmaceutical Compositions

Disclosed herein, in some embodiments, are methods, compositions and kits for use of the modified cells, including pharmaceutical compositions, therapeutic methods, and methods of administration. Although the descriptions of pharmaceutical compositions provided herein are principally directed to pharmaceutical compositions which are suitable for administration to humans, it will be understood by the skilled artisan that such compositions are generally suitable for administration to any animals.

In some embodiments, a pharmaceutical composition comprising a modified autologous host cell as described herein is provided. The modified autologous host cell is genetically engineered to comprise an integrated wild-type JAK2 coding sequence that has replaced a mutant JAK2^{V617F} allele in the genome. The modified host cell of the disclosure herein may be formulated using one or more excipients to, e.g.: (1) increase stability; (2) alter the biodistribution (e.g., target the cell line to specific tissues or cell types); (3) alter the release profile of an encoded therapeutic factor.

Formulations of the present disclosure can include, without limitation, saline, liposomes, lipid nanoparticles, polymers, peptides, proteins, and combinations thereof. Formulations of the pharmaceutical compositions described herein may be prepared by any method known or hereafter developed in the art of pharmacology. As used herein the term “pharmaceutical composition” refers to compositions including at least one active ingredient (e.g., a modified host cell) and optionally one or more pharmaceutically acceptable excipients. Pharmaceutical compositions of the present disclosure may be sterile.

Relative amounts of the active ingredient (e.g., the modified host cell), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between 0.5 and 50%, between 1-30%, between 5-80%, or at least 80% (w/w) active ingredient.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy. 21st Edition, A. R. Gennaro, Lippincott, Williams & Wilkins, Baltimore, MD, 2006: incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

### Dosing and Administration

The modified host cells of the present disclosure included in the pharmaceutical compositions described above may be administered by any delivery route, systemic delivery or local delivery, which results in a therapeutically effective outcome. These include, but are not limited to, enteral, gastroenteral, epidural, oral, transdermal, intracerebral, intracerebroventricular, epicutaneous, intradermal, subcutaneous, nasal, intravenous, intra-arterial, intramuscular, intracardiac, intraosseous, intrathecal, intraparenchymal, intraperitoneal, intravesical, intravitreal, intracavernous), interstitial, intra-abdominal, intralymphatic, intramedullary, intrapulmonary, intraspinal, intrasynovial, intrathecal, intratubular, parenteral, percutaneous, periarticular, peridural, perineural, periodontal, rectal, soft tissue, and topical. In particular embodiments, the cells are administered intravenously.

In some embodiments, a subject will undergo a conditioning regime before cell transplantation. For example, before hematopoietic stem cell transplantation, a subject may undergo myeloablative therapy, non-myeloablative therapy or reduced intensity conditioning to prevent rejection of the stem cell transplant even if the stem cell originated from the same subject. The conditioning regime may involve administration of cytotoxic agents. The conditioning regime may also include immunosuppression, antibodies, and irradiation. Other possible conditioning regimens include antibody-mediated conditioning (see, e.g., Czechowicz et al., 318(5854) Science 1296-9 (2007); Palchaudari et al., 34(7) Nature Biotechnology 738-745 (2016): Chhabra et al., 10:8(351) Science Translational Medicine 351ra105 (2016)) and CAR T-mediated conditioning (see, e.g., Arai et al., 26(5) Molecular Therapy 1181-1197 (2018); each of which is hereby incorporated by reference in its entirety). For example, conditioning needs to be used to create space in the brain for microglia derived from engineered hematopoietic stem cells (HSCs) to migrate in to deliver the protein of interest (as in recent gene therapy trials for ALD and MLD). The conditioning regimen is also designed to create niche “space” to allow the transplanted cells to have a place in the body to engraft and proliferate. In HSC transplantation, for example, the conditioning regimen creates niche space in the bone marrow for the transplanted HSCs to engraft. Without a conditioning regimen, the transplanted HSCs cannot engraft.

Certain aspects of the present disclosure are directed to methods of providing pharmaceutical compositions including the modified host cell of the present disclosure to target tissues of mammalian subjects, by contacting target tissues with pharmaceutical compositions including the modified host cell under conditions such that they are substantially retained in such target tissues. In some embodiments, pharmaceutical compositions including the modified host cell include one or more cell penetration agents, although “naked” formulations (such as without cell penetration agents or other agents) are also contemplated, with or without pharmaceutically acceptable excipients.

The present disclosure additionally provides methods of administering modified host cells in accordance with the disclosure to a subject in need thereof. The pharmaceutical compositions including the modified host cell, and compositions of the present disclosure may be administered to a subject using any amount and any route of administration effective for preventing, treating, or managing the disorder, e.g., polycythemia vera. The exact amount required will vary from subject to subject, depending on the species, age, and general condition of the subject, the severity of the disease, the particular composition, its mode of administration, its mode of activity, and the like. The subject may be a human, a mammal, or an animal. The specific therapeutically or prophylactically effective dose level for any particular individual will depend upon a variety of factors including the disorder being treated and the severity of the disorder; the activity of the specific payload employed: the specific composition employed: the age, body weight, general health, sex and diet of the patient; the time of administration, route of administration; the duration of the treatment: drugs used in combination or coincidental with the specific modified host cell employed; and like factors well known in the medical arts.

In certain embodiments, modified host cell pharmaceutical compositions in accordance with the present disclosure may be administered at dosage levels sufficient to deliver from, e.g., about 1×10⁴ to 1×10⁵, 1×10⁵ to 1×10⁶, 1×10⁶ to 1×10⁷, or more modified cells to the subject, or any amount sufficient to obtain the desired therapeutic or prophylactic, effect. The desired dosage of the modified host cells of the present disclosure may be administered one time or multiple times. In some embodiments, delivery of the modified host cell to a subject provides a therapeutic effect for at least 1 month, 2 months, 3 months, 4 months, 5 months, 6 months, 7 months, 8 months, 9 months, 10 months, 11 months, 1 year, 13 months. 14 months, 15 months, 16 months, 17 months, 18 months. 19 months, 20 months, 20 months, 21 months, 22 months, 23 months, 2 years. 3 years, 4 years, 5 years, 6 years, 7 years, 8 years, 9 years, 10 years or more than 10 years.

The modified host cells may be used in combination with one or more other therapeutic, prophylactic, research or diagnostic agents, or medical procedures, either sequentially or concurrently. In general, each agent will be administered at a dose and/or on a time schedule determined for that agent.

Use of a modified mammalian host cell according to the present disclosure for treatment of polycythemia or another myeloproliferative disorder is also encompassed by the disclosure.

The present disclosure also contemplates kits comprising compositions or components of the present disclosure, e.g., sgRNA, Cas9, RNPs, i53, and/or homologous templates, as well as, optionally, reagents for, e.g., the introduction of the components into cells. The kits can also comprise one or more containers or vials, as well as instructions for using the compositions in order to modify cells and treat subjects according to the methods described herein.

### 6. Examples

The present disclosure will be described in greater detail by way of specific examples. The following examples are offered for illustrative purposes only, and are not intended to limit the disclosure in any manner. Those of skill in the art will readily recognize a variety of noncritical parameters which can be changed or modified to yield essentially the same results.

### Example 1. Genomic Editing Based Correction of JAK2 Mutation Underlying Polycythemia Vera

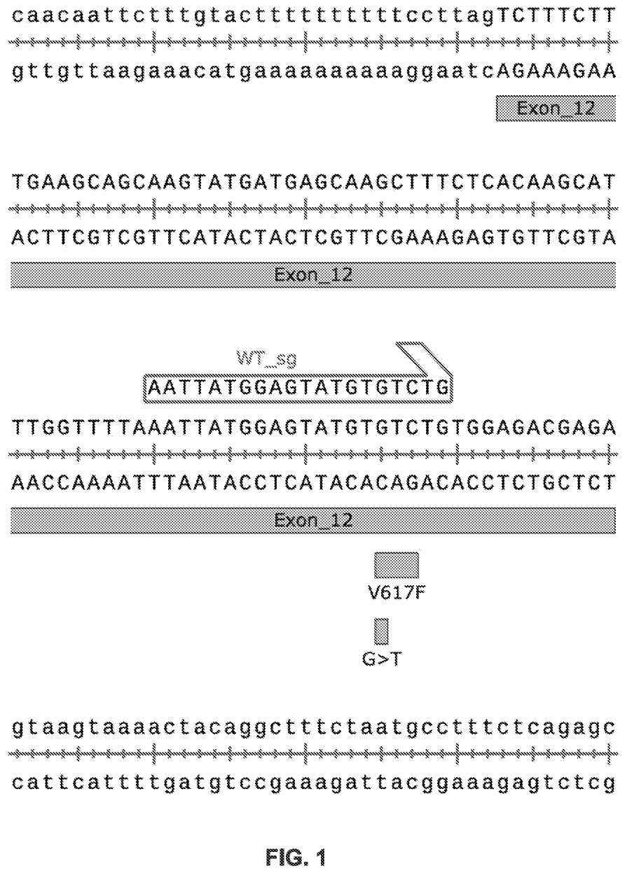

For the SpCas9 ortholog (which is the most heavily characterized Cas9 ortholog and the one used in most current ex vivo editing workflows), a nearby NGG PAM sequence is required for a guide sequence to overlap the JAK2^{V617F} mutation (G>T missense mutation) (FIG. 1). We identified such a PAM sequence overlapping the mutation, raising the prospect of using the high-fidelity variant SpCas9 (Vakulskas, et al., Nature Medicine, 2018) to specifically cut the mutant allele while preserving the WT allele.

Because WT HSPCs are more readily available than patient cells, we first tested the ability to create indels using guides overlapping the mutated basepair (corresponding to the WT or the mutant sequence). These experiments were performed under the assumption that the ability to specifically cut the WT sequence and not the mutant sequence in V T HSPCs can serve as a proxy for the ability to specifically cut the mutant allele and not the WT allele in mutant HSPCs.

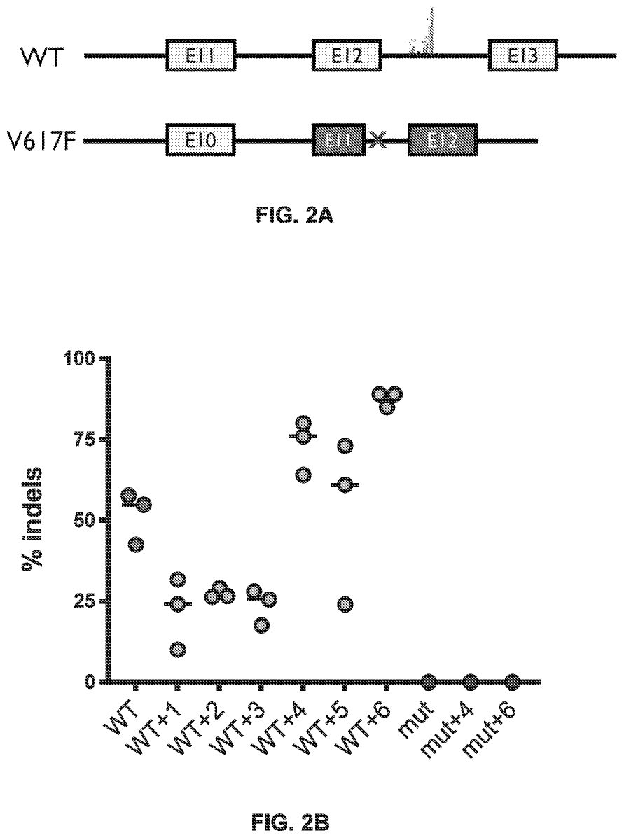

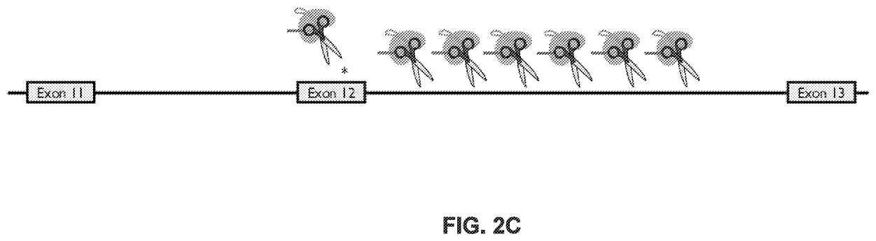

Toward this end, we designed WT- and mutant-overlapping guides and found that our WT guide created ˜50% indels whereas the mutant guide sequence (distinguished by only a single altered nucleotide 5 bp from the PAM) induced no detectable cutting (FIG. 2A). This % as done using hi-fi SpCas9 RNP pre-complexed with guide RNA and delivered by electroporation, as previously described (Bak, et al., Nature Protocols, 2016).

Because this mutation is gain-of-function, it may be necessary to obtain close to 100% KO/correction of the mutant allele. Therefore, to induce a higher frequency of mutant-specific deletion, we designed gRNAs in the following intron (i.e., the intron between exons 12 and 13) that may be paired as a two-guide system (FIGS. 2B-2C). We found that by combining Cas9 RNP pre-complexed with WT guide and guides 4 and 6, we were able to induce a high degree of deletions between the predicted cleavage sites of each gRNA (FIG. 2B).

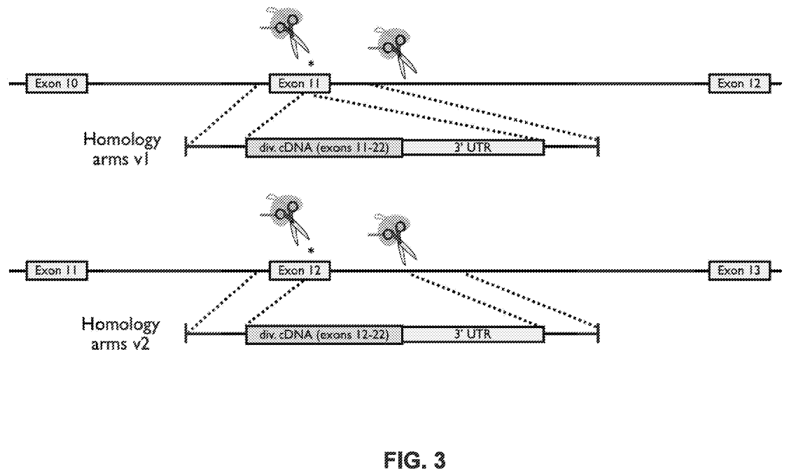

Proceeding with our two best 2-guide systems (WT gRNA paired with sg4 or sg6), we next screened homology arms that best mediated integration of a custom cassette. Version 1 (top panel of FIG. 3) is the typical homology arm scheme used for homology-directed repair following a Cas9-induced DNA break—with homology arms immediately flanking the disease-causing mutation to essentially “wedge” in the integration cassette. Version 2 (bottom panel of FIG. 3) is a homology arm scheme where the left HA corresponds to the DNA sequence immediately upstream of the leftmost guide's cut site while the right HA corresponds to the DNA sequence immediately downstream of the rightmost guide's cut site.

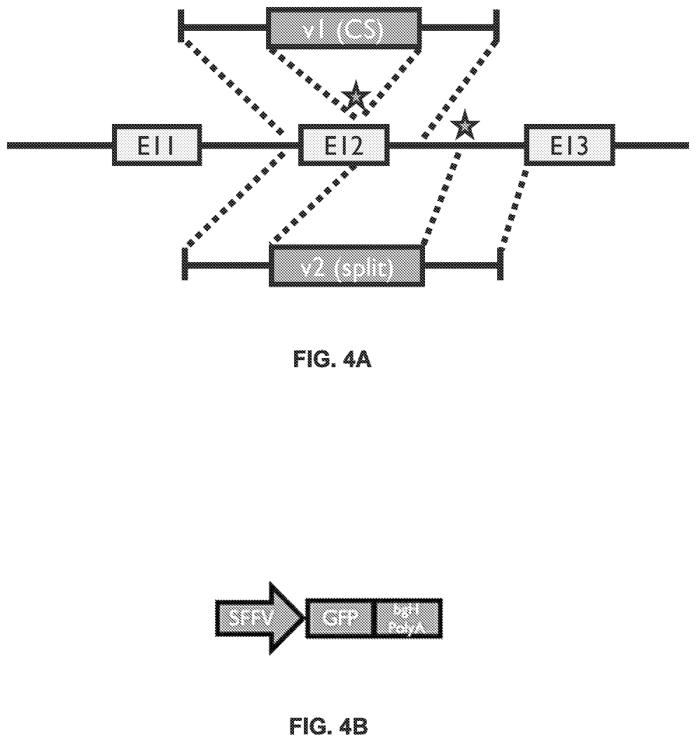

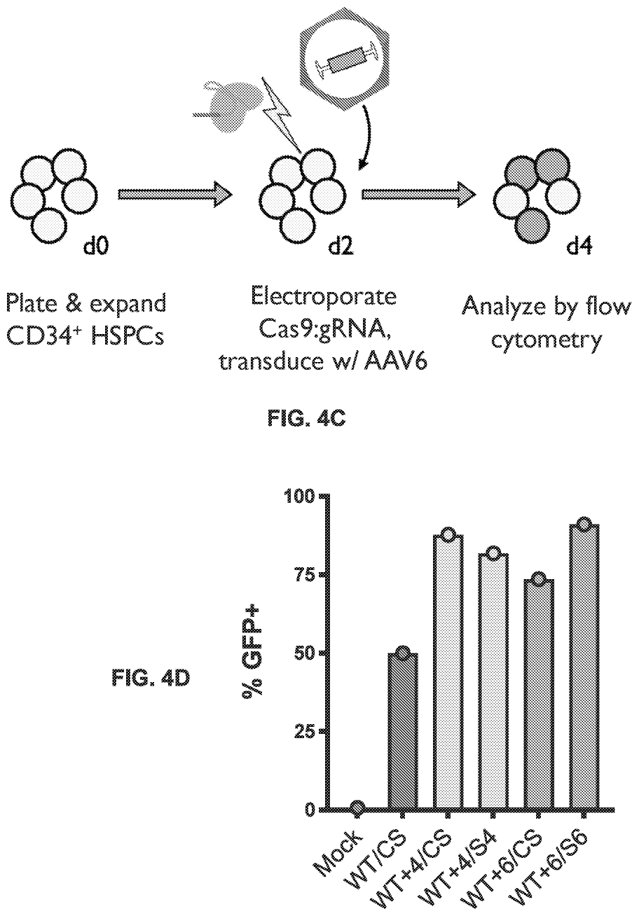

To test these alternative homology arm schemes for their ability to mediate targeted integration, we designed constitutive SFFV-GFP-polyA vectors (FIGS. 4A-4B) to rapidly identify editing frequencies by flow cytometry (FIG. 4C). In doing so, we found that all 2-guide editing schemes were more effective than the single WT gRNA paired with the cut-site (“CS”) integration scheme (FIG. 4D).

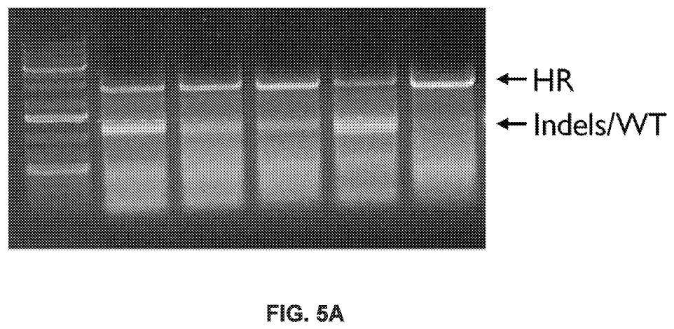

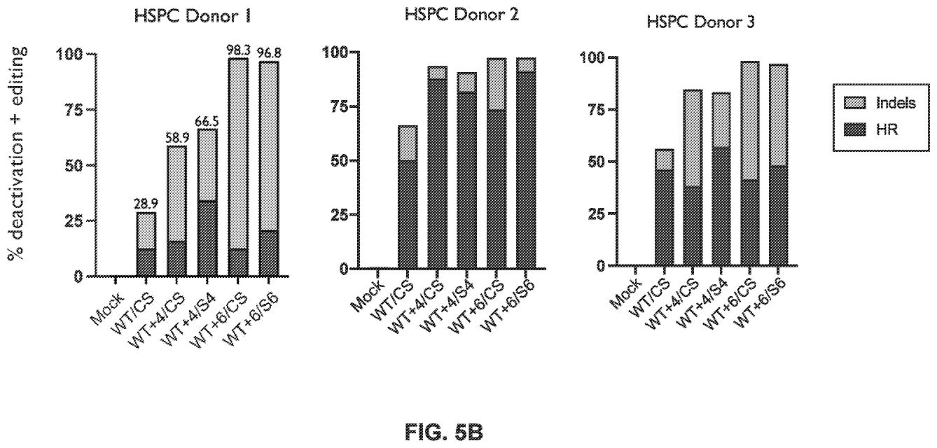

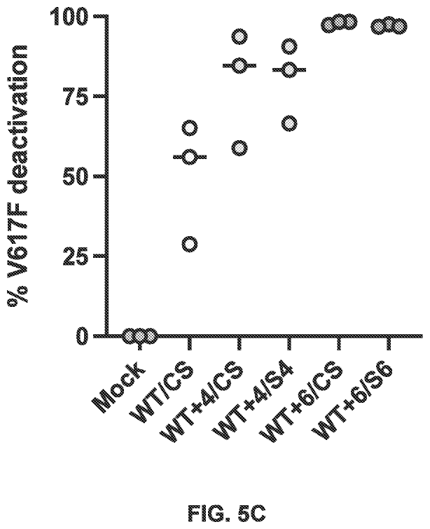

Next, we used ddPCR to quantify editing frequencies of our SFFV-GFP-polyA vectors in three separate healthy HSPC donors. In addition to this, we used primers outside of the homology arms to amplify the edited locus and excised the band that did not undergo knock-in (FIG. 5A). We then Sanger sequenced these bands and quantified indels using ICE (ice.synthego.com). Percentages of homologous recombination (HR)+indel alleles are shown above each column in FIG. 5B and summarized in FIG. 5C.

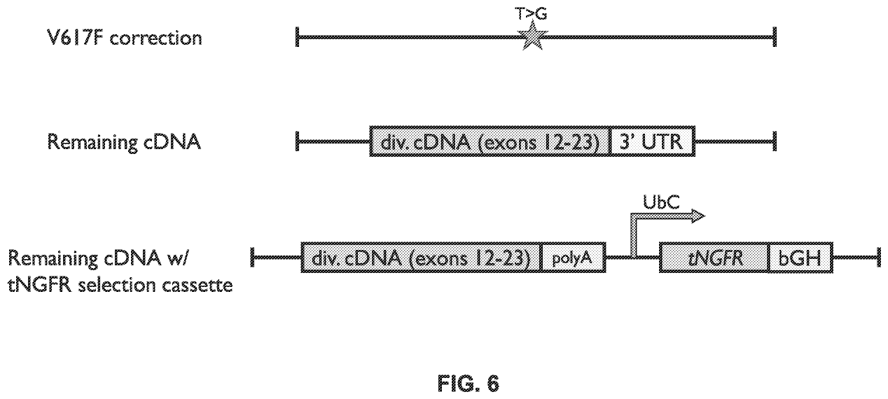

We can generate clinical correction vectors that either: 1) directly correct the V617F mutation; 2) introduce the remaining JAK2 cDNA following the mutation, or 3) the remaining JAK2 cDNA followed by a constitutive PGK-tNGFR selection cassette (FIG. 6). These vectors use a mutant-overlapping guide along with sg6 and have homology arms that immediately flank the V617F mutation. We first test these in WT HSPCs and quantify editing by ddPCR for both HR and indel alleles. To determine the success of the tNGFR cassette, we determine HR and indel frequencies before and after sorting tNGFR+ HSPCs by FACS.

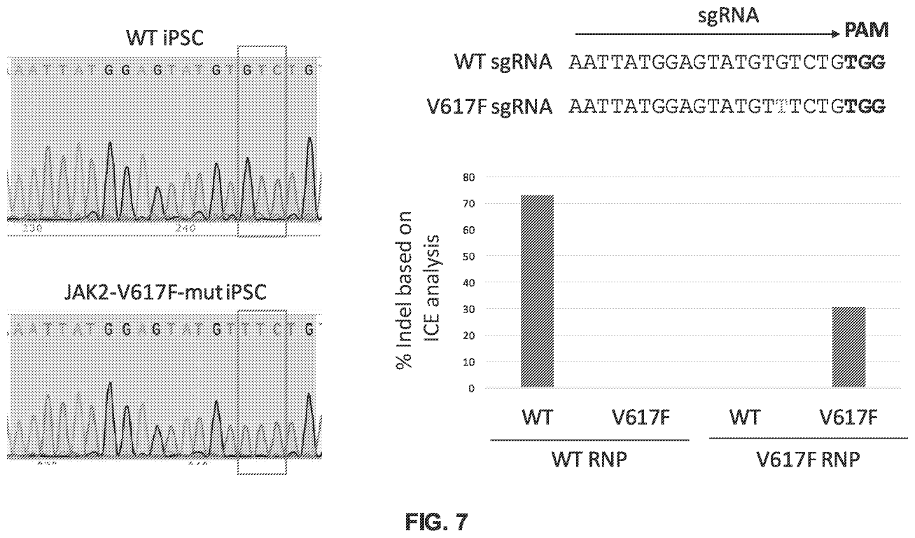

All experiments described above were performed using human WT CD34+ HSPCs. We then used a gRNA specific to the JAK2 WT sequence as a proxy for how efficiently we may target this region in JAK2 mutant cells. While FIG. 2B shows that the mutant-specific gRNA is unable to cleave WT cells at detectable frequencies, we had yet to show that the mutant gRNA has equivalent activity and specificity in the presence of mutant alleles. Therefore, we created a homozygous JAK2^{V617F} cell line and then electroporated HiFi Cas9 complexed with either JAK2 WT- or mutant-specific gRNAs into both WT and mutant iPSC lines. Indeed, we found that both gRNAs—while only distinguished by a single nucleotide—display extreme specificity to the intended allele (FIG. 7). No cleavage was observed when targeting the WT cell line with the mutant gRNA and no cleavage was observed when targeting the mutant cell line with the WT gRNA.

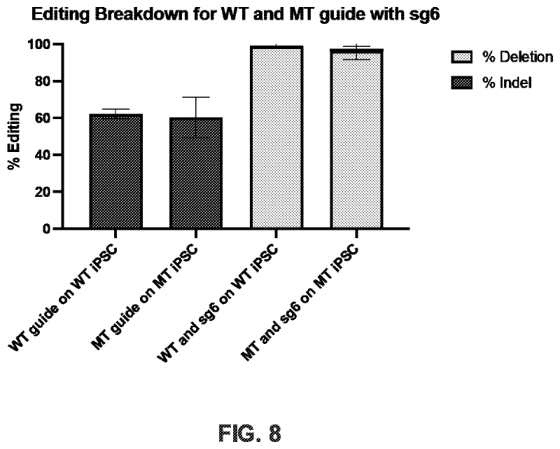

We next tested whether the dual-gRNA strategy is able to boost JAK2 deactivation above the single mutant-specific gRNA approach in WT and mutant iPSC lines. Indeed, when paired with sg6, editing efficiency for both WT gRNA in WT iPSCs and mutant gRNA in mutant iPSCs was dramatically improved (FIG. 8).

### Materials and Methods

### AAV6 Vector Design, Production, and Purification

All AAV6 vectors were cloned into the pAAV-MCS plasmid (Agilent Technologies, Santa Clara, CA, USA), which contains inverted terminal repeats (ITRs) derived from AAV2. Gibson Assembly Mastennix (New England Biolabs, Ipswich, MA, USA) was used for the creation of each vector as per manufacturer's instructions. Few modifications were made to the production of AAV6 vectors as described¹. 293T cells (Life Technologies, Carlsbad, CA, USA) were seeded in five 15 cm² dishes with 17×10⁶ cells per plate. 24 h later, each dish was transfected with a standard polyethylenimine (PEI) transfection of 6 μg ITR-containing plasmid and 22 μg pDGM6 (gift from David Russell, University of Washington, Seattle, WA, USA), which contains the AAV6 cap genes, AAV2 rep genes, and Ad5 helper genes. After a 48-72 h incubation, cells were purified using AAVPro Purification Kits (All Serotypes)(Takara Bio USA, Mountain View, CA, USA) as per manufacturer's instructions. AAV6 vectors were titered using ddPCR to measure number of vector genomes per μL as previously described².

### Culturing of CD34⁺ HSPCs

Human CD34⁺ HSPCs were cultured as previously described³⁻⁸. Healthy donor CD34⁺ HSPCs were sourced from fresh cord blood (generously provided by Binns Family program for Cord Blood Research), frozen cord blood, and Plerixafor- and/or G-CSF-mobilized peripheral blood (AilCells, Alameda, CA, USA and STEMCELL Technologies, Vancouver, Canada). Polycythcmia vera patient-derived HSPCs were sourced from discarded bone marrow aspirates and phlebotomy samples under an IRB-approved protocol. CD34⁺ HSPCs were cultured at 1×10⁵ cells/mL in Stem Span SFEM II (STEMCELL Technologies, Vancouver, Canada) base medium supplemented with stem cell factor (SCF)(100 ng/mL), thrombopoietin (TPO)(100 ng/mL), FLT3-ligand (100 ng/mL), IL-6 (100 ng/mL), UM171 (35 nM), streptomycin (20 mg/mL), and penicillin (20 U/mL). The cell incubator conditions were 37° C., 5% CO₂, and 5% O₂.

### Genome Editing of CD34⁺ HSPCs

Chemically modified Cas9 sgRNAs were purchased from Symthego (Menlo Park, CA, USA) and TriLink BioTechnologies (San Diego, CA, USA) and were purified by high-performance liquid chromatography (HPLC). The sgRNA modifications added were the 2′-O-methyl-3′-phosphorothioate at the three terminal nucleotides of the 5′ and 3′ ends described previously⁹. The target sequences for sgRNAs were as follows: JAK2-WTsg: 5′-AATTATGGAGTATGTGTCTG-3′; JAK2-V617Fsg: 5′-AATTATGGAGTATGTTTCTG-3′; JAK2intron-sg1: 5′-ACGAGAGTAAGTAAAACTAC-3′; JAK2intron-sg2: 5′-AAAAACAGATGCTCTGAGAA-3′; JAK2intron-sg3: 5′-TATATAGAAAATTCAGTTTC-3′: JAK2intron-sg4: 5′-TCAGTTTCAGGATCACAGCT-3: JAK2intron-sg5: 5′-AGTGTAAACTATAATTTAAC-3′; and JAK2intron-sg6: 5′-TTTGAAACTGAAAACACTGT-3′. All hi-fidelity variant¹⁰ Cas9 protein (SpyFi) used was purchased from Aldevron, LLC (Fargo, ND, USA). The RNPs were complexed at a Cas9:sgRNA molar ratio of 1:2.5 at 25° C. for 10 min prior to electroporation. When two-guide targeting strategies were used, each guide was separately pre-complexed with Cas9 at the stated molar ratio at half the standard amount used for single-guide targeting. CD34⁺ cells were resuspended in P3 buffer (Lonza, Basel, Switzerland) with complexed RNPs and electroporated using the Lonza 4D Nucleofector (program DZ-100). Cells were plated at 1×10⁵ cells/mL following electroporation in the cytokine-supplemented media described previously. Immediately following electroporation. AAV6 was supplied to the cells at 5×10³ vector genomes/cell based on titers determined by ddPCR.

### Gene Targeting Analysis by Flow Cytometry

For targeting analysis by flow cytometry, CD34⁺ HSPCs were harvested at d5 and erythrocytes at d16 post-targeting. Cells were analyzed for viability using Ghost Dye Red 780 (Tonbo Biosciences, San Diego, CA, USA) and reporter expression was assessed using the FACS Aria I1 system (BD Biosciences, San Jose, CA, USA). The data was subsequently analyzed using FlowJo (FlowJo LLC, Ashland, OR, UJSA).

In Vitro Differentiation of CD34⁺ HSPCs into Erythrocytes

Following targeting, HSPCs derived from healthy donors or polycythemia vera patients were cultured for 14-16 d at 37° C. and 5% CO₂ in SFEM II medium (STEMCELL Technologies, Vancouver, Canada) as previously described^{11, 12}. SFEMII base medium was supplemented with 100 U/mL penicillin-streptomycin, 10 ng/mL SCF, 1 ng/mL IL-3 (PepmTech, Rocky Hill, NJ, USA). 3 U/mL erythropoietin (eBiosciences, San Diego, CA, USA), 200 μg/mL transferrin (Sigma-Aldrich, St. Louis, MO, USA), 3% antibody serum (heat-inactivated from Atlanta Biologicals, Flowery Branch, GA, USA), 2% human plasma (derived from umbilical cord blood), 10 μg/mL insulin (Sigma-Aldrich, St. Louis, MO, USA), and 3 U/mL heparin (Sigma-Aldrich, St. Louis, MO, USA). In the first phase, d 0-7 (d0 being 2 d post-targeting) of differentiation, cells were cultured at 1×10⁵ cells/mL. In the second phase, d7-10, cells were maintained at 1×10⁵ cells/mL, and IL-3 was removed from the culture. In the third phase, d11-16, cells were cultured at 1×10⁶ cells/mL, and transferrin was increased to 1 mg/mL within the culture medium.

### Immunophenotyping of Differentiated Erythrocytes

HSPCs subjected to the above erythrocyte differentiation were analyzed at d14 for erythrocyte lineage-specific markers using a FACS Aria II (BD Biosciences, San Jose, CA, USA). Edited and non edited cells were analyzed by flow cytometry using the following antibodies: hCD45 V450 (H130. BD Biosciences, San Jose, CA, USA), CD34 APC (561; BioLegend, San Diego, CA, USA), CD71 PE-Cy7 (OKT9; Affymetris, Santa Clara, CA, USA), and CD235a PE (GPA)(GA-R2: BD Biosciences, San Jose, CA, USA).

### Indel Frequency Analysis by TIDE

2-4 d post-targeting. HSPCs were harvested and QuickExtract DNA extraction solution (Epicentre, Madison, WI, USA) was used to collect gDNA. Primers were then used to amplify the region surrounding the predicted cut site and/or deletion. PCR reactions were then run on a 1% agarose gel and appropriate bands were cut and gel-extracted using a GeneJET Gel Extraction Kit (Thermo Fisher Scientific, Waltham, MA, USA) according to manufacturer's instructions. Gel-extracted amplicons were then Sanger sequenced and resulting chromatograms were used as input for indel frequency analysis by TIDE as previously described¹³.

Allelic Targeting Analysis by ddPCR

2-4 d post-targeting, HSPCs were harvested and QuickExtract DNA extraction solution (Epicentre, Madison, WI, USA) was used to collect gDNA. gDNA was then digested using BamHI-HF as per manufacturer's instructions (New England Biolabs, Ipswich, MA, USA). The percentage of targeted alleles within a cell population was measured by ddPCR using the following reaction mixture: 1-4 μL of digested gDNA input, 10 μL ddPCR SuperMix for Probes (No dUTP)(Bio-Rad, Hercules, CA, USA), primer/probes (13.6 ratio; Integrated DNA Technologies, Coralville, Iowa, USA), volume up to 20 μL with H₂O. ddPCR droplet were then generated following the manufacturer's instructions (Bio-Rad, Hercules, CA, USA): 20 μL of ddPCR reaction, 70 μL droplet generation oil, and 40 μL of droplet sample. Thermocycler (Bio-Rad, Hercules, CA, USA) settings were as follows: 1. 98° C. (10 min), 2. 94° C. (30 s), 3. 57.3° C. (30 s), 4. 72° C. (1.75 min)(return to step 2×40-50 cycles), 5. 98° C. (10 min). Analysis of droplet samples was done using the QX200 Droplet Digital PCR System (Bio-Rad, Hercules. CA, USA). To determine percentage of alleles targeted, the number of Poisson-corrected integrant copies/mL were divided by the number of Poisson-corrected reference DNA copies/mL.

### REFERENCES

1. Khan, I. F., Hirata, R. K. & Russell, D. W. AAV-mediated gene targeting methods for human cells. Nat Protoc 6, 482-501 (2011).
2. Aurnhammer, C. et al. Universal real-time PCR for the detection and quantification of adeno-associated virus serotype 2-derived inverted terminal repeat sequences. Hum Gene Ther Methods 23, 18-28 (2012).
3. Dever, D. P. et al. CRISPR/Cas9 beta-globin gene targeting in human haematopoietic stem cells. Nature 539, 384-389 (2016).
4. Charlesworth, C. T. et al. Priming Human Repopulating Hematopoietic Stem and Progenitor Cells for Cas9/sgRNA Gene Targeting. Mol Ther Nucleic Acids 12, 89-104 (2018).
5. Bak, R. O., Dever, D. P. & Porteus, M. H. CRISPR/Cas9 genome editing in human hematopoietic stem cells. Nat Protoc 13, 358-376 (2018).
6. Bak, R. O. et al. Multiplexed genetic engineering of human hematopoietic stem and progenitor cells using CRISPR/Cas9 and AAV6. Elife 6 (2017).
7. Cromer, M. K. et al. Global Transcriptional Response to CRISPR/Cas9-AAV6-Based Genome Editing in CD34(+) Hematopoietic Stem and Progenitor Cells. Mol Ther 26, 2431-2442 (2018).
8. Bak, R. O. & Porteus. M. H. CRISPR-Mediated Integration of Large Gene Cassettes Using AAV Donor Vectors. Cell Rep 20, 750-756 (2017).
9. Hendel, A. et al. Chemically modified guide RNAs enhance CRISPR-Cas genome editing in human primary cells. Nat Biotechnol 33, 985-989 (2015).
10. Vakulskas, C. A. et al. A high-fidelity Cas9 mutant delivered as a ribonucleoprotein complex enables efficient gene editing in human hematopoietic stem and progenitor cells. Nat Med 24, 1216-1224 (2018).
11. Dulmovits, B. M. et al. Pomalidomide reverses gamma-globin silencing through the transcriptional reprogramming of adult hematopoietic progenitors. Blood 127, 1481-1492 (2016).
12. Hu, J. et al. Isolation and functional characterization of human erythroblasts at distinct stages: implications for understanding of normal and disordered erythropoiesis in vivo. Blood 121, 3246-3253 (2013).
13. Brinkman, E. K., Chen, T., Amendola, M. & van Steensel, B. Easy quantitative assessment of genome editing by sequence trace decomposition. Nucleic Acids Res 42, e168 (2014).

### 7. Exemplary Embodiments

Exemplary embodiments provided in accordance with the presently disclosed subject matter include, but are not limited to, the claims and the following embodiments:

1. A method of genetically modifying a hematopoietic stem and progenitor cell (HSPC) comprising a JAK2^{V617F} mutation from a subject, the method comprising:
introducing into the HSPC an RNA-guided nuclease, a donor template, and a mutation-specific guide RNA that specifically hybridizes to a mutant JAK2 polynucleotide comprising a JAK2^{V617F} mutation, but does not hybridize to a wild-type JAK2 polynucleotide lacking the JAK2^{V617F} mutation; wherein
the donor template comprises a corrective JAK2 nucleotide sequence that comprises a wild-type sequence at the position of the JAK2^{V617F} mutation, flanked by a first homology arm corresponding to a JAK2 genomic sequence located upstream of the JAK2^{V617F} mutation and a second homology arm corresponding to a JAK2 genomic sequence located downstream of the JAK2^{V617F} mutation; wherein
the RNA-guided nuclease cleaves a mutant JAK2^{V617F} gene in the genome of the cell but does not cleave a wild-type JAK2 gene in the genome of the cell; and wherein
the cleaved mutant JAK2^{V617F} gene is modified by integrating the corrective JAK2 nucleotide sequence into the genome by homology directed repair (HDR), thereby eliminating the JAK2^{V617F} mutation from the genome and generating a genetically modified HSPC.

2. The method of embodiment 1, wherein the method further comprises isolating the HSPC from the subject prior to introducing the RNA-guided nuclease, the donor template, and the mutation-specific guide RNA into the cell.

3. The method of embodiment 1 or 2, wherein the method further comprises introducing into the HSPC a second guide RNA comprising a target site located within an intron in the JAK2 gene.

4. The method of embodiment 3, wherein the intron is located between exons 12 and 13 of the JAK2 gene.

5. The method of embodiment 3 or 4, wherein the second homology arm corresponds to a JAK2 genomic sequence located downstream of the target site of the second guide RNA.

6. The method of any one of embodiments 1 to 5, wherein the first homology arm comprises the nucleotide sequence of SEQ ID NO: 1 or a subsequence thereof, or a sequence comprising at least about 70%, 75%, 80%, 85%, 90%, 95%, 96%, 97%, 98%, 99%, or more identity to SEQ ID NO:1 or a subsequence thereof.

7. The method of any one of embodiments 1 to 6, wherein the second homology arm comprises the nucleotide sequence of SEQ ID NO:2 or a subsequence thereof, or a sequence comprising at least about 70%, 75%, 80%, 85%, 90%, 95%, 96%, 97%, 98%, 99%, or more identity to SEQ ID NO:2 or a subsequence thereof.

8. The method of any one of embodiments 1 to 7, wherein the corrective JAK2 nucleotide sequence comprises a portion of exon 12 downstream of the site corresponding to the JAK2^{V617F} mutation, and all of exons 13-23 of the wild-type JAK2 gene.

9. The method of embodiment 8, wherein the donor template comprises SEQ ID NO:4, or a sequence comprising at least about 70%, 75%, 80%, 85%, 90%, 95%, 96%, 97%, 98%, 99%, or more identity to SEQ ID NO:4.

10. The method of any one of embodiments 1 to 9, wherein the corrective JAK2 nucleotide sequence comprises a JAK2 3′ UTR.

11. The method of embodiment 10, wherein the JAK2 3 UTR comprises the sequence of SEQ ID NO:5 or a subsequence thereof, or a sequence comprising at least about 70%, 75%, 80%, 85%, 90%, 95%, 96%, 97%, 98%, 99%, or more identity to SEQ ID NO:5 or a subsequence thereof.

12. The method of any one of embodiments 1 to 11, wherein the mutation-specific guide RNA specifically hybridizes to a polynucleotide comprising the nucleotide sequence of SEQ ID NO:9, and does not specifically hybridize to a polynucleotide comprising the nucleotide sequence of SEQ ID NO:8.

13. The method of any one of embodiments 3 to 12, wherein the target sequence of the second guide RNA comprises the nucleotide sequence of any one of SEQ ID NOS:10-15.

14. The method of embodiment 13, wherein the target sequence of the second guide RNA comprises the nucleotide sequence of SEQ ID NO: 15.

15. The method of any one of embodiments 1 to 14, wherein the mutation-specific and/or second guide RNA comprises one or more 2′-O-methyl-3′-phosphorothioate (MS) modifications.

16. The method of embodiment 15, wherein the one or more 2′-O-methyl-3′-phosphorothioate (MS) modifications are present at the three terminal nucleotides of the 5′ and 3′ ends of the guide RNA.

17. The method of any one of embodiments 1 to 16, wherein the RNA-guided nuclease is Cas9.

18. The method of embodiment 17, wherein the Cas9 is a High Fidelity Cas9.

19. The method of any one of embodiments 1 to 18, wherein the mutation-specific and/or second guide RNA and the RNA-guided nuclease are introduced into the HSPC as a ribonucleoprotein (RNP) complex by electroporation.

20. The method of any one of embodiments 1 to 19, wherein the donor template is introduced into the HSPC using a recombinant adeno-associated virus (rAAV) vector.

21. The method of embodiment 20, wherein the rAAV vector is an AAV6 vector.

22. The method of any one of embodiments 1 to 21, wherein the method reduces the proliferation and/or erythropoietic differentiation of the genetically modified HSPC as compared to an HSPC into which the mutation-specific guide RNA, the RNA-guided nuclease, and/or the donor template has not been introduced.

23. The method of any one of embodiments 1 to 22, wherein the subject has polycythemia very (PV).

24. The method of embodiment 23, wherein the genetically modified HSPC is reintroduced into the subject.

25. The method of embodiment 24, wherein the reintroduction of the genetically modified HSPC ameliorates one or more symptoms of PV.

26. The method of any one of embodiments 1 to 25, wherein the subject is a human.

27. A genetically modified HSPC comprising a corrective JAK2 nucleotide sequence, wherein the genetically modified HSPC is generated using the method of any one of embodiments 1 to 26.

28. A donor template comprising a homology region comprising SEQ ID NO:1 or SEQ ID NO:2 or a subsequence thereof, or a nucleotide sequence comprising at least about 70%, 75%, 80%, 85%, 90%, 95%, 96%, 97%, 98%, 99% or more identity to SEQ ID NO:1 or SEQ ID NO:2 or a subsequence thereof.

29. A donor template comprising a nucleotide sequence comprising SEQ ID NO:7 or a subsequence thereof, or a sequence comprising at least about 70%, 75%, 80%, 85%, 90%, 95%, 96%, 97%, 98%, 99%, or more identity to SEQ ID NO:7 or a subsequence thereof.

30. A transgene comprising a corrective JAK2 nucleotide sequence, wherein the nucleotide sequence comprises the sequence of SEQ ID NO:4 or a nucleotide sequence comprising at least about 70%, 75%, 80%, 85%, 90%, 95%, 96%, 97%, 98%, 99% or more identity to SEQ ID NO:4.

31. A guide RNA that specifically hybridizes to a polynucleotide comprising the nucleotide sequence of SEQ ID NO:9, but does not specifically hybridize to a polynucleotide comprising the nucleotide sequence of SEQ ID NO:8.

32. A guide RNA comprising a target sequence comprising any one of SEQ ID NOS: 10-15, or a sequence comprising 1, 2, or 3 mismatches with any one of SEQ ID NOS:10-15.

33. The guide RNA of embodiment 32, wherein the target sequence comprises the nucleotide sequence of SEQ ID NO:15.

34. A ribonucleoprotein (RNP) complex comprising: (a) an RNA-guided nuclease; and (b) the guide RNA of embodiment 31 and/or the guide RNA of embodiment 32 or 33.

35. An HSPC comprising the donor template of embodiment 28 or 29, the transgene of embodiment 30, the guide RNA of any one of embodiments 31-33, and/or the RNP complex of embodiment 34.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A method of genetically modifying a hematopoietic stem and progenitor cell (HSPC) comprising a JAK2^{V617F} mutation from a subject, the method comprising:
introducing into the HSPC an RNA-guided nuclease, a donor template, and a mutation-specific guide RNA that specifically hybridizes to a mutant JAK2 polynucleotide comprising a JAK^{v617F} mutation, but does not hybridize to a wild-type JAK2 polynucleotide lacking the JAK^{V617F} mutation; wherein
the donor template comprises a corrective JAK2^{V617F} nucleotide sequence that comprises a wild-type sequence at the position of the JAK2^{V617F} mutation, flanked by a first homology arm corresponding to a JAK2 genomic sequence located upstream of the JAK2^{V617F} mutation and a second homology arm corresponding to a JAK2 genomic sequence located downstream of the JAK2^{V617F} mutation; wherein
the RNA-guided nuclease cleaves a mutant JAK2^{V617F} gene in the genome of the cell but does not cleave a wild-type JAK2 gene in the genome of the cell; and wherein
the cleaved mutant JAK2^{V617F} gene is modified by integrating the corrective JAK2 nucleotide sequence into the genome by homology directed repair (HDR), thereby eliminating the JAK2^{V617F} mutation from the genome and generating a genetically modified HSPC.

2. (canceled)

3. The method of claim 1, wherein the method further comprises introducing into the HSPC a second guide RNA comprising a target site located within an intron in the JAK2 gene.

4. (canceled)

5. (canceled)

6. The method of claim 1, wherein the first homology arm comprises the nucleotide sequence of SEQ ID NO: 1 or a subsequence thereof, or a sequence comprising at least about 70%, 75%, 80%, 85%, 90%, 95%, 96%, 97%, 98%, 99%, or more identity to SEQ ID NO: 1 or a subsequence thereof, wherein the subsequence is 500 to 600 base pairs in length.

7. The method of claim 1, wherein the second homology arm comprises the nucleotide sequence of SEQ ID NO:2 or a subsequence thereof, or a sequence comprising at least about 70%, 75%, 80%, 85%, 90%, 95%, 96%, 97%, 98%, 99%, or more identity to SEQ ID NO:2 or a subsequence thereof, wherein the subsequence is 550 to 650 base pairs in length.

8. The method of claim 1, wherein the corrective JAK2 nucleotide sequence comprises a portion of exon 12 downstream of the site corresponding to the JAK2^{V617F} mutation, and all of exons 13-23 of the wild-type JAK2 gene.

9. The method of claim 8, wherein the donor template comprises SEQ ID NO:4, or a sequence comprising at least about 70%, 75%, 80%, 85%, 90%, 95%, 96%, 97%, 98%, 99%, or more identity to SEQ ID NO:4.

10. The method of claim 1, wherein the corrective JAK2 nucleotide sequence comprises a JAK2 3′ UTR.

11. (canceled)

12. The method of claim 1, wherein the mutation-specific guide RNA specifically hybridizes to a polynucleotide comprising the nucleotide sequence of SEQ ID NO:9, and does not specifically hybridize to a polynucleotide comprising the nucleotide sequence of SEQ ID NO:8.

13. The method of claim 3, wherein the target sequence of the second guide RNA comprises the nucleotide sequence of any one of SEQ ID NOS:10-15.

14. (canceled)

15. The method of claim 1, wherein the mutation-specific RNA comprises one or more 2′-O-methyl-3′-phosphorothioate (MS) modifications.

16. (canceled)

17. The method of claim 1, wherein the RNA-guided nuclease is Cas9.

18. The method of claim 17, wherein the Cas9 is a High Fidelity Cas9.

19. The method of claim 1, wherein the mutation-specific guide RNA and the RNA-guided nuclease are introduced into the HSPC as a ribonucleoprotein (RNP) complex by electroporation.

20. The method of claim 1, wherein the donor template is introduced into the HSPC using a recombinant adeno-associated virus (rAAV) vector.

21. The method of claim 20, wherein the rAAV vector is an AAV6 vector.

22. The method of claim 1, wherein the method reduces the proliferation and/or erythropoietic differentiation of the genetically modified HSPC as compared to an HSPC into which the mutation-specific guide RNA, the RNA-guided nuclease, and/or the donor template has not been introduced.

23. The method of claim 1, wherein the subject has polycythemia vera (PV).

24. The method of claim 23, wherein the genetically modified HSPC is reintroduced into the subject.

25. The method of claim 24, wherein the reintroduction of the genetically modified HSPC ameliorates one or more symptoms of PV.

26. The method of claim 1, wherein the subject is a human.

27. A genetically modified HSPC comprising a corrective JAK2 nucleotide sequence, wherein the genetically modified HSPC is generated using the method of claim 1.

28. A donor template comprising a homology region comprising SEQ ID NO: 1 or SEQ ID NO:2 or a subsequence thereof, or a nucleotide sequence comprising at least about 70%, 75%, 80%, 85%, 90%, 95%, 96%, 97%, 98%, 99% or more identity to SEQ ID NO: 1 or SEQ ID NO:2 or a subsequence thereof, wherein the subsequence is 500 to 650 base pairs in length.

29. (canceled)

30. A transgene comprising a corrective JAK2 nucleotide sequence, wherein the nucleotide sequence comprises the sequence of SEQ ID NO:4 or a nucleotide sequence comprising at least about 70%, 75%, 80%, 85%, 90%, 95%, 96%, 97%, 98%, 99% or more identity to SEQ ID NO:4.

31. (canceled)

32. (canceled)

33. (canceled)

34. (canceled)

35. An HSPC comprising the donor template of claim 28.

36. The method of claim 1, wherein the donor template further comprises a selection cassette comprising a marker gene operably linked to a promoter.

37. The method of claim 36, wherein the selection cassette comprises a PGK-tNGFR selection cassette.


SEQUENCE LISTING (18 sequences)

SEQ ID NO: 1 - DNA, 900 bp, synthetic construct
  misc_feature 1..900: note = Description: JAK2-integrating left homology arm (900 bp sequence upstream of mutation-specific guide RNA target site)
  source 1..900: mol_type = other DNA
catatactgtggccttgattttcaaacttggctccccagagctttatgggttacacagaggctcctctacaattacatttatttgaaaaaaaaacaaaaaaccaagtttcaaaggcactgtactaaggaacatgcttagtgaaatctagatgcctctgggcatcatccagagatgctagctggcagaccccgtggccagttgccagagctctgaacatagcaagatatgatacttacaatatcttaaagggtgttggtgttagaagatgatgtgaaaagttcaagccaaagagttgttctaatccagaataccacagtattggtgattgtgattcactaatcatacccaggggttctagtcacagtttagttgaattagagtgatgttaaaactatgctagtatcctgacacagatgtcgtgatattttatctgcacattcttaattctttagcaagtgttatttaaaggctacatccatctacctcagtttcctatatctatctctgacatctacctctagttgtacttctgtcctctatttcaggtgttatgggtcaagcctgtttgactggcattattcatgattcctgtaccactcttgctctctctcactttgatctccatattccaggcttacacaggggtttcctcagaacgttgatggcagttgcaggtccatataaagggaccaaagcacattgtatcctcatctatagtcatgctgaaagtaggagaaagtgcatctttattatggcagagagaattttctgaactatttatggacaacagtcaaacaacaattctttgtacttttttttttccttagtctttctttgaagcagcaagtatgatgagcaagctttctcacaagcatttggttttaaattatggagtatgt

SEQ ID NO: 2 - DNA, 1057 bp, synthetic construct
  misc_feature 1..1057: note = Description: JAK2-integrating right homology arm (1057 bp sequence downstream of mutation-specific guide RNA target site)
  source 1..1057: mol_type = other DNA
ctgtggagacgagagtaagtaaaactacaggctttctaatgcctttctcagagcatctgtttttgtttatatagaaaattcagtttcaggatcacagctaggtgtcagtgtaaactataatttaacaggagttaagtatttttgaaactgaaaacactgtaggactattcagttatatcttgtgaaaaaggaaagcaatgaagttaaaagtagaaggttacaatgcccaaacaatagagtattatagtaaacaaatgtctataaaacattttgtgttcatgatagcaaaagagattatggcaggttcaacataacattggaataactggccttttcagtacaaacttatctggaattatgaagacaaagcatataaatgatacacttaatttttaatggaactgacagaaatgattatgttgatatgatactagatatattttttggctaaatttaggtgttcacagaaactactaaaagtataaatcgtaccccatgctttaatactatacaggcatgcctcattttattgcaccttgctttattgtgcttcttagatattgtattttttacatattgaaggtttacggcaacccagtgtctagcaactctgtcagcaacattttcccaacagcatgtgctcatttcatgtctctgtgtcatattttggtaattctagcaacatttcaaactttaaaaaaatcatatggtgatctgtgatcagtaatctttaatgctagtattgtaattattctggggtgtcccaaacagagagaatataagaaggcaaacttcatagatgaatgcagtgtgtgctctgactgctccatcaatgagccattcctctgtctctctccctctcctggggcctccctattccctgagacaacaatattaaaattaggccatttaataatcacacaatggtctttaaatgttcaagtgaaaggaagattcacccgtctctcactttaaatggaaagctagaaatgaagcagcaaacgctgatgtggaagctgcagcagttatccagaagatctagcaaagaccattgatg

SEQ ID NO: 3 - DNA, 900 bp, synthetic construct
  misc_feature 1..900: note = Description: JAK2-integrating right homology arm (900 bp sequence downstream of intron-specific guide RNA target site)
  source 1..900: mol_type = other DNA
tgtaggactattcagttatatcttgtgaaaaaggaaagcaatgaagttaaaagtagaaggttacaatgcccaaacaatagagtattatagtaaacaaatgtctataaaacattttgtgttcatgatagcaaaagagattatggcaggttcaacataacattggaataactggccttttcagtacaaacttatctggaattatgaagacaaagcatataaatgatacacttaatttttaatggaactgacagaaatgattatgttgatatgatactagatatattttttggctaaatttaggtgttcacagaaactactaaaagtataaatcgtaccccatgctttaatactatacaggcatgcctcattttattgcaccttgctttattgtgcttcttagatattgtattttttacatattgaaggtttacggcaacccagtgtctagcaactctgtcagcaacattttcccaacagcatgtgctcatttcatgtctctgtgtcatattttggtaattctagcaacatttcaaactttaaaaaaatcatatggtgatctgtgatcagtaatctttaatgctagtattgtaattattctggggtgtcccaaacagagagaatataagaaggcaaacttcatagatgaatgcagtgtgtgctctgactgctccatcaatgagccattcctctgtctctctccctctcctggggcctccctattccctgagacaacaatattaaaattaggccatttaataatcacacaatggtctttaaatgttcaagtgaaaggaagattcacccgtctctcactttaaatggaaagctagaaatgaagcagcaaacgctgatgtggaagctgcagcagttatccagaagatctagcaaagaccattgatg

SEQ ID NO: 4 - DNA, 1548 bp, synthetic construct
  misc_feature 1..1548: note = Description: Remaining diverged JAK2 cDNA (middle of exon 12-23)
  source 1..1548: mol_type = other DNA
gtgtgcggcgatgaaaacatcctcgtgcaagaattcgtgaagttcggcagcctggacacctacctcaagaaaaacaagaactgcatcaacatcctgtggaagctggaggtggccaagcaactggcctgggctatgcacttcctggaggagaatacactgatccacggcaacgtgtgcgctaagaacatcctcctgattcgggaggaggatcggaaaaccggcaaccccccctttattaagctgagcgaccccggaatcagcatcaccgtgctgcccaaagatatcctgcaagaacggatcccctgggtgccccccgagtgtatcgagaaccccaagaacctgaacctggccaccgataagtggagcttcggcacaaccctgtgggagatttgtagcggcggcgacaagcccctgagcgccctcgacagccagcggaaactgcagttctacgaggaccggcaccaactgcccgcccccaaatgggccgagctggccaatctgatcaacaactgcatggactacgagcccgactttcggcccagctttcgggctattatccgggacctgaatagcctgttcacccccgactacgagctgctgaccgagaacgatatgctgcccaacatgcggatcggcgctctcggcttcagcggcgctttcgaggatagagaccccacccaattcgaggaacggcacctgaagttcctgcaacagctgggaaaaggcaacttcggcagcgtcgaaatgtgtagatacgatcccctgcaagataataccggcgaagtcgtggccgtgaagaaactgcaacacagcaccgaggaacatctgcgggatttcgagcgggagatcgagattctcaagagcctgcaacacgataatatcgtgaaatataaaggcgtctgttatagcgccggcagacggaacctgaagctgatcatggagtacctgccctacggcagcctgcgggattacctgcagaagcacaaggagagaatcgaccatatcaagctgctccaatataccagccaaatctgtaaaggcatggaatacctgggcaccaagcggtacattcatcgggacctcgccacccggaacatcctggtcgaaaatgaaaatcgggtgaagatcggcgacttcggcctgacaaaggtgctgccccaggataaggagtattacaaggtgaaggagcccggcgagagccctatcttttggtacgcccccgagagcctcaccgaatccaaattcagcgtcgctagcgacgtgtggtccttcggcgtcgtgctctacgagctgtttacctatatcgaaaaaagcaagagcccccccgccgagttcatgcggatgatcggaaacgataagcagggccaaatgattgtctttcacctgatcgagctgctgaaaaacaacggccggctgccccggcccgacggctgtcccgacgaaatttacatgattatgaccgagtgttggaataacaacgtgaaccagcggcctagcttccgggacctggccctgcgggtcgaccagatccgggacaatatggccggc

SEQ ID NO: 5 - DNA, 1387 bp, synthetic construct
  misc_feature 1..1387: note = Description: JAK2 3' UTR
  source 1..1387: mol_type = other DNA
aagaaatgaccttcattctgagaccaaagtagatttacagaacaaagttttatatttcacattgctgtggactattattacatatatcattattatataaatcatgatgctagccagcaaagatgtgaaaatatctgctcaaaactttcaaagtttagtaagtttttcttcatgaggccaccagtaaaagacattaatgagaattccttagcaaggattttgtaagaagtttcttaaacattgtcagttaacatcactcttgtctggcaaaagaaaaaaaatagactttttcaactcagctttttgagacctgaaaaaattattatgtaaattttgcaatgttaaagatgcacagaatatgtatgtatagtttttaccacagtggatgtataataccttggcatcttgtgtgatgttttacacacatgagggctggtgttcattaatactgttttctaatttttccatagttaatctataattaattacttcactatacaaacaaattaagatgttcagataattgaataagtacctttgtgtccttgttcatttatatcgctggccagcattataagcaggtgtatacttttagcttgtagttccatgtactgtaaatatttttcacataaagggaacaaatgtctagttttatttgtataggaaatttccctgaccctaaataatacattttgaaatgaaacaagcttacaaagatataatctattttattatggtttcccttgtatctatttgtggtgaatgtgttttttaaatggaactatctccaaatttttctaagactactatgaacagttttcttttaaaattttgagattaagaatgccaggaatattgtcatcctttgagctgctgactgccaataacattcttcgatctctgggatttatgctcatgaactaaatttaagcttaagccataaaatagattagattgttttttaaaaatggatagctcattaagaagtgcagcaggttaagaattttttcctaaagactgtatatttgaggggtttcagaattttgcattgcagtcatagaagagatttatttcctttttagaggggaaatgaggtaaataagtaaaaaagtatgcttgttaattttattcaagaatgccagtagaaaattcataacgtgtatctttaagaaaaatgagcatacatcttaaatcttttcaattaagtataaggggttgttcgttgttgtcatttgttatagtgctactccactttagacaccatagctaaaataaaatatggtgggttttgtgtgtgtgtgtgtgtgtgtgtgtgtgtgtgtgtgtgtgttatttatacaaaacttaaaatacttgctgttttgattaaaaagaaaatagtttctta

SEQ ID NO: 6 - DNA, 2416 bp, synthetic construct
  misc_feature 1..2416: note = Description: polyA-UbC-tNGFR-bGH (edited cell selection cassette)
  source 1..2416: mol_type = other DNA
atgctttatttgtgaaatttgtgatgctattgctttatttgtaaccattataagctgcaataaacaagttaacaacaacaattgcattcattttatgtttcaggttcagggggaggtgtgggaggttttttaaagttatccctaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacggtaccgagcatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccatgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcgggtgtggtgaccacagtgatgggcagctcccagcccgtggtgacccgaggcaccaccgacaacctcatccctgtctattgctccatcctggctgctgtggttgtgggtcttgtggcctacatagccttcaagaggtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 7 - DNA, 4401 bp, synthetic construct
  misc_feature 1..4401: note = Description: JAK2-V617F SNP correction donor (includes silent mutations surrounding Cas9 cut site to eliminate cutting following homologous recombination)
  source 1..4401: mol_type = other DNA
agatgtagtgaacctcaagacagatctgaagaaattaagaatgcagcagttagggagaaagaaatggattatatgaaagagggattaagtggcatggaaggcaaaatgagaaagacccaactcatatctgagctctataaggagacaatagatagaattagccagaatggtaaagacaagcatcttcatgtgagtaacaggaaacagagtggccaggtaacttgactaacattacaaatctaataaatagtagaccttggatttgaacacaagcagccttactgcagagtgcaggctttcaacaattactttgtaaagatatgcaagaaatactagtattagtagtaatagctgatacttaatgcacatttactatgtgtcatggactgtgctaagtgcttttatgtatttattcacttattgatcataacaatcctatgggaaaatagttattattctcatttttcagatgaagaaaccacgacagctgcagcacagagattaaatagcctgtacaaggtcatacaagtggtaataagagaaagtcagatattctgcctctgcagaccacattctctagtgtgctagtaggaaaggcaaagaaaatgattcccatgtaagcaaatgtaggtaaacactgactctatttttaaaaagtgacaattatagcccattcaggagatttcaaaaccgagtagagccaaaatacacttaattcattaatccttgttttctgtctcttattactatttaaggaagcagaataaaagcattaatttggagtcttaaatctggatttagattctaaggaaaatacttgcttatggatttaaaaaaattcttcctcattgaatgtattttcttgttcctacttcgttctccatctttactcattcttttcttttacctttttctcttgaagaatgaaagccttggccaaggcacttttacaaagatttttaaaggcgtacgaagagaagtaggagactacggtcaactgcatgaaacagaagttcttttaaaagttctggataaagcacacagaaactattcagaggtgtgtatgttctttatattgttcatgtagtttatgctgtttaaagatgtgctctcatatgcatacaacgtactcatgtgtgcagcttttcaaaattgtaatttttaaatgtgtcaaggacttttctgaggatacattcttgtggggctatagaattacagggtttgaaaattaccggattaaaaaaattaattccaaactaattatgtttagcattatgttaggagtgttattacatttctgcatatactgtggccttgattttcaaacttggctccccagagctttatgggttacacagaggctcctctacaattacatttatttgaaaaaaaaacaaaaaaccaagtttcaaaggcactgtactaaggaacatgcttagtgaaatctagatgcctctgggcatcatccagagatgctagctggcagaccccgtggccagttgccagagctctgaacatagcaagatatgatacttacaatatcttaaagggtgttggtgttagaagatgatgtgaaaagttcaagccaaagagttgttctaatccagaataccacagtattggtgattgtgattcactaatcatacccaggggttctagtcacagtttagttgaattagagtgatgttaaaactatgctagtatcctgacacagatgtcgtgatattttatctgcacattcttaattctttagcaagtgttatttaaaggctacatccatctacctcagtttcctatatctatctctgacatctacctctagttgtacttctgtcctctatttcaggtgttatgggtcaagcctgtttgactggcattattcatgattcctgtaccactcttgctctctctcactttgatctccatattccaggcttacacaggggtttcctcagaacgttgatggcagttgcaggtccatataaagggaccaaagcacattgtatcctcatctatagtcatgctgaaagtaggagaaagtgcatctttattatggcagagagaattttctgaactatttatggacaacagtcaaacaacaattctttgtacttttttttttccttagtctttctttgaagcagcaagtatgatgagcaagctttctcacaagcatttggttttaaactacggcgtgtgcgtgtgcggagacgagagtaagtaaaactacaggctttctaatgcctttctcagagcatctgtttttgtttatatagaaaattcagtttcaggatcacagctaggtgtcagtgtaaactataatttaacaggagttaagtatttttgaaactgaaaacactgtaggactattcagttatatcttgtgaaaaaggaaagcaatgaagttaaaagtagaaggttacaatgcccaaacaatagagtattatagtaaacaaatgtctataaaacattttgtgttcatgatagcaaaagagattatggcaggttcaacataacattggaataactggccttttcagtacaaacttatctggaattatgaagacaaagcatataaatgatacacttaatttttaatggaactgacagaaatgattatgttgatatgatactagatatattttttggctaaatttaggtgttcacagaaactactaaaagtataaatcgtaccccatgctttaatactatacaggcatgcctcattttattgcaccttgctttattgtgcttcttagatattgtattttttacatattgaaggtttacggcaacccagtgtctagcaactctgtcagcaacattttcccaacagcatgtgctcatttcatgtctctgtgtcatattttggtaattctagcaacatttcaaactttaaaaaaatcatatggtgatctgtgatcagtaatctttaatgctagtattgtaattattctggggtgtcccaaacagagagaatataagaaggcaaacttcatagatgaatgcagtgtgtgctctgactgctccatcaatgagccattcctctgtctctctccctctcctggggcctccctattccctgagacaacaatattaaaattaggccatttaataatcacacaatggtctttaaatgttcaagtgaaaggaagattcacccgtctctcactttaaatggaaagctagaaatgaagcagcaaacgctgatgtggaagctgcagcagttatccagaagatctagcaaagaccattgatgaagatggcttagagaggaaggcatgctgaaagccaagacaagctgaaagctaggcctcttgcactaaaaaggcaagttatgaatccaaaggaaaagtccttgaaggaaactgaaagtgctactcctgtgagcacataaattacaagaaagtgaaacagtcttattgatgatacagagaaagttctagatagaagatcaaaccagtcacaacattcccttgagccaaagcccaatctagagcaagatcttaatgctcttaaattctatgaaggctgagagaggtgaggaagctgcagaagaaaagttggaagctagcagaggttggttcatgaggtttaagaaaagaagccatgtgcacagcataagtgcaaggtgaagcagcaaatgctgatgtggaagctacagcaagttatccagaagatctagcaaggaccattgatgaagatggtgacactaacaaacagattttcaaatgtaaacaaaacagccttctgttggaagaatatgccatctaggacttccacagctagagaggagaagtcagtgcttgccttcaaagcttcgaaggacaggctgaccctcttgctaagggccaatgccattggtgatttgaaattcaaaccagcgctcatttaccattccaaaaatcccagggctctgcgaattatgctaagtctactctgcctgtgctctatacatggaacaagaaagcctagatgacagcacatctgtttatagcatggtttattgactattttaagcccattgttgagacctgctcagaaaaaaagattgctttcaaagtattactgatcattgacaatgcacctggtcacccaagagctctgatggagatgtacaaggatattcatgttattttcaggcctggtgacacaacatccattctgtagcccatggatcaaggagttatttcaactttcaagtcttattatctaagaaatacattttgtaaggctaagctgccatagatagtgattcctctggtggatctgggtaaagtaagtagaaaaacttatggaaaggattcaccattctagatgccaggaatagcattcatggttcatggaaggagatcaaaatggcagcattaataggaatttggaaaaagttcattctaaccc

SEQ ID NO: 8 - DNA, 20 bp, synthetic construct
  misc_feature 1..20: note = Description: JAK2-WTsg
  source 1..20: mol_type = other DNA
aattatggagtatgtgtctg

SEQ ID NO: 9 - DNA, 20 bp, synthetic construct
  misc_feature 1..20: note = Description: JAK2-V617Fsg
  source 1..20: mol_type = other DNA
aattatggagtatgtttctg

SEQ ID NO: 10 - DNA, 20 bp, synthetic construct
  misc_feature 1..20: note = Description: JAK2intron-sg1
  source 1..20: mol_type = other DNA
acgagagtaagtaaaactac

SEQ ID NO: 11 - DNA, 20 bp, synthetic construct
  misc_feature 1..20: note = Description: JAK2intron-sg2
  source 1..20: mol_type = other DNA
aaaaacagatgctctgagaa

SEQ ID NO: 12 - DNA, 20 bp, synthetic construct
  misc_feature 1..20: note = Description: JAK2intron-sg3
  source 1..20: mol_type = other DNA
tatatagaaaattcagtttc

SEQ ID NO: 13 - DNA, 20 bp, synthetic construct
  misc_feature 1..20: note = Description: JAK2intron-sg4
  source 1..20: mol_type = other DNA
tcagtttcaggatcacagct

SEQ ID NO: 14 - DNA, 20 bp, synthetic construct
  misc_feature 1..20: note = Description: JAK2intron-sg5
  source 1..20: mol_type = other DNA
agtgtaaactataatttaac

SEQ ID NO: 15 - DNA, 20 bp, synthetic construct
  misc_feature 1..20: note = Description: JAK2intron-sg6
  source 1..20: mol_type = other DNA
tttgaaactgaaaacactgt

SEQ ID NO: 16 - DNA, 1660 bp, synthetic construct
  misc_feature 1..1660: note = Description: SFFV-GFP-bGH integration cassette
  source 1..1660: mol_type = other DNA
tctagaccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggaccggtgccaccatggccccaaagaagaagcggaaggtcggtatccacggagtcccagcagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtacc

SEQ ID NO: 17 - DNA, 23 bp, synthetic construct
  misc_feature 1..23: note = Description: JAK2-WTsg with PAM sequence
  source 1..23: mol_type = other DNA
aattatggagtatgtgtctgtgg

SEQ ID NO: 18 - DNA, 23 bp, synthetic construct
  misc_feature 1..23: note = Description: JAK2-V617Fsg with PAM sequence
  source 1..23: mol_type = other DNA
aattatggagtatgtttctgtgg